(19) United States
(12) Patent Application Publication
(10) Pub. No.: US 2009/0253709 A1
(43) Pub. Date: Oct. 8, 2009

(54) SYNTHESIS, TELOMERASE INHIBITION AND CYTOTOXIC STUDIES ON 2,7-DISUBSTITUTED ANTHRAQUINONE DERIVATIVES

(75) Inventor: Hsu-Shan HUANG, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: NATIONAL DEFENSE MEDICAL CENTER, Taipei (TW)

(21) Appl. No.: 12/192,468

(22) Filed: Aug. 15, 2008

(30) Foreign Application Priority Data
Apr. 2, 2008 (TW) 097112088

(51) Int. Cl. C07C 50/18; A61K 31/167; A61K 31/4025; C07D 401/12; C07D 403/12; A61K 31/4545; A61K 31/496; A61P 35/04

(52) U.S. Cl. 514/252.11; 552/297; 514/616; 514/422; 548/523; 546/190; 514/316; 544/357

(57) ABSTRACT

An series of 2,7-disubstituted anthraquinone derivatives including a formula I are provided.

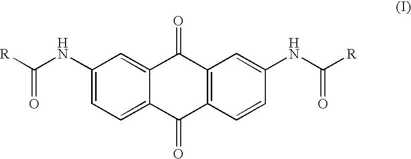

R is a first substituted group selected from a group consisting of a hydrogen, an amino group, a nitro group, a hydroxyl group, a C₁-C₁₂ alkyl group, a C₁-C₁₂ alkyl halide group (—(CH2)ₙX), a C₃-C₁₂ cycloalkyl group, a benzyl group, a C₁-C₁₂ alkylamino group, a C₅-C₁₂ nitrocycloalkyl group and a heterocyclic group, n satisfies 1≦n≦12 and X is an atom selected from a group consisting of a fluoride (F), a chloride (Cl), a bromide (Br) and an iodine (I). The preparation method of the 2,7-disubstituted anthraquinone derivatives includes the steps of acetylating 2,7-diaminoanthraquinone to be one 2,7-disubstituted anthraquinone derivative, which can be further aminated to be another 2,7-disubstituted anthraquinone derivative.

### FIELD OF THE INVENTION

The present invention relates to an anthraquinone compound having cytotoxicity and the preparation method thereof. In particular, the present invention relates to an anthraquinone compound which achieves the purpose of cytotoxicity by inhibiting the telomerase activity, and the preparation method thereof.

### BACKGROUND OF THE INVENTION

Telomere has a structure of ribonucleoprotein. In eukaryotes, telomere mainly is located in the end of chromosome, and has the portions of deoxyribonucleic acid (DNA) and signal strand DNA. In human, telomere is mainly composed of the guanine (G)-rich repeated sequence, TTAGGG, and the length of telomere is about 15,000 base pairs (bp). However, the composition of sequence and repeated numbers would be diverse within the species. The main function of telomere lies in protecting the end of chromosome so as to prevent degradation, recombination and end-to-end fusion of telomere. In the normal somatic cells, the end of chromosome would reduce a partial ribonucleic acid (RNA) primer because of each replication, and telomere would shorten 50 to 60 bp after each mitosis. When telomere shortens to a particular level, the cell will be proceed apoptosis. This is cell's end-replication problem.

In the most eukaryotes, the replication and maintenance of telomere must depend on a specific reverse transcriptase nominated as telomerase. Telomerase, which is a ribonucleoprotein containing RNA and protein in the meanwhile, can recognize the G-rich protruded single strand of telomere, and telomere is replicated by utilizing the RNA sequence of telomerase as the template. For instance, the RNA template in human's telomerase has a nucleic acid sequence, 5′-CCCUAA-3′. This nucleic acid sequence can be utilized to replicate human's telomere sequence. In accordance with the researches, telomerase activity can be determined in continuously divided cells (such as hematopoietic cells, germline cells and stem cells, etc.), and telomerase activity disappears after mitosis. Accordingly, telomerase activity cannot be detected in human's normal somatic cells.

Telomerase activity exists in 85 to 90% human carcinoma cells. Telomerase mainly includes two parts. One is the subunit having reverse transcriptase, which is also nominated as human telomerase reverse transcriptase (hTERT). The other is the RNA template, which includes 11 basic sequences (i.e. AATCCC) complementary to the G-rich sequences of telomere. This RNA template is also nominated as human telomerase RNA component (hTR). In the telomere replication, the RNA portion (i.e. hTR) of telomerase is being the template for synthesizing the repeated sequence of telomere, and the telomere replication is proceeded by the protein subunit (i.e. hTERT) of telomere having reverse transcriptase activity. The length of telomere is elongated or maintained by this mechanism, so as to maintain the carcinoma cells without senescence. This is the reason that carcinoma cells differentiate continuously and do not proceed apoptosis normally as normal cells (Yamashita et al., 2005).

In the normal physiological condition, the quadruplex structure is naturally formed in the G-rich single strand of the end of chromosome. The quadruplex structure includes two parts. One is a small loop composed of a nucleic acid sequence (i.e. TTA), and the other is the guanine-tetrad structure composed of four guanines by cyclic hydrogen bonding. The repeated sequence of telomere would not be elongated by telomerase because of stabilizing the quadruplex structure and inhibiting the function of quadraplex structure to the complementary single-strand RNA (i.e. CCCTAA).

Some compounds, such as anthraquinone derivatives, quinoacridine derivatives, phenanthroline derivatives, substituted triazine derivatives and acridine derivatives, etc., stabilize the quadraplex structure by the interaction between these compounds and the quinine-tetrad, so as to achieve the inhibition of telomerase Among these, anthraquinone derivatives includes heterocyclic anthraquinone derivatives (Peng et al., 2005) and 2,7-diamino-substituted anthraquinone derivatives. The synthetic method of anthraquinone derivative has published as the aforementioned references. Therefore, the research in inhibiting telomerase mainly includes two aspects. One is to inhibit the main expressing regulation factor, hTERT, of telomerase so as to achieve the inhibition of telomerase. The other is to stabilize the specific G-quadruplex structure formed by telomerase itself, and arrest the function between telomerase and telomere, so as to achieve the inhibition effect. However, attempts regarding to the abovementioned aspects are not yet achieved the completely satisfied results.

It is therefore attempted by the applicant to deal with the above situation encountered in the prior art.

### SUMMARY OF THE INVENTION

In accordance with one aspect of the present invention, a 2,7-disubstituted anthraquinone derivative is provided. The 2,7-disubstituted anthraquinone derivative includes a formula I as follows:

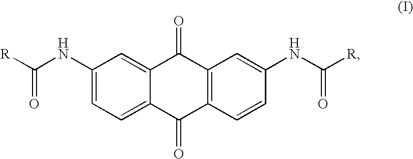

wherein R is a first substituted group selected from a group consisting of a hydrogen, an amino group, a nitro group, a hydroxyl group, a C₁-C₁₂ alkyl group, a C₁-C₁₂ alkyl halide group (—(CH2)ₙX), a C₃-C₁₂ cycloalkyl group, a benzyl group, a C₁-C₁₂ alkylamino group, a C₅-C₁₂ nitrocycloalkyl group and a heterocyclic group, n satisfies 1≦n≦12 and X is an atom selected from a group consisting of a fluoride (F), a chloride (Cl), a bromide (Br) and an iodine (I).

Preferably, the C₁-C₁₂ alkyl group is one of a linear C₁-C₁₂ alkyl group and a branched C₁-C₁₂ alkyl group.

Preferably, the C₁-C₁₂ alkyl group includes a methyl group, an ethyl group, a propyl group, a butyl group, an isobutyl group, a pentyl group, an isopentyl group, a heptyl group, an isoheptyl group, an octyl group, an iso-octyl group and a linear alkyl group with a C<5 branched alkyl group.

Preferably, the C₁-C₁₂ alkyl halide group includes a methylhalide group, an ethylhalide group, a propylhalide group, a butylhalide group, an isobutylhalide group, a pentylhalide group, an isopentylhalide group, a heptylhalide group, an isoheptylhalide group, an octylhalide group, an iso-octylhalide group and a linear alkylhalide group with a C<5 branched alkyl group.

Preferably, the benzyl group has a para-position, a meta position and an ortho-position, at least one of which is bounded with a second substituted group selected from a group consisting of a hydrogen, a linear C₁-C₃ alkyl group, a branched C₃ alkyl group and a C₁-C₃ alkylamino group.

Preferably, the C₃-C₁₂ cycloalkyl group includes a cyclopropyl group, a cyclopentyl group, a cyclohexyl group, a cycloheptyl group, a cyclo-octyl group, an alkyl ring with a C<5 branched alkyl group, a cyclopropylhalide group, a cyclopentylhalide group, a cyclohexylhalide group, a cycloheptylhalide group, a cyclo-octylhalide group and an alkyl ring with a branched alkylhalide group.

Preferably, each one of the C₃-C₁₂ cycloalkyl group has a para-position, a meta position and an ortho-position, at least one of which is bounded with a third substituted group selected from a group consisting of a hydrogen, a branched C₁-C₃ alkyl group and a C₁-C₃ alkylamino group.

Preferably, the C₁-C₁₂ alkylamino group is one of a linear C₁-C₁₂ alkylamino group and a branched C₁-C₁₂ alkylamino group.

Preferably, each one of the C₅-C₁₂ nitrocycloalkyl group has a para-position, a meta position and an ortho-position, at least one of which is bounded with a fourth substituted group selected from a group consisting of a hydrogen, an amino group, a nitro group, a hydroxyl group, a C₁-C₅ alkyl group, a C<3 branched alkyl group, a C₃-C₅ cycloalkoxyl group, an alkylamino group, a hydroxylhalide group, a C₁-C₅ alkylhalide group, a C<3 branched alkylhalide group, and a C₃-C₅ cycloalkoxylhalide group.

Preferably, each one of the heterocyclic group has a para-position, a meta position and an ortho-position, at least one of which is bounded with a fifth substituted group selected from a group consisting of a hydrogen, an amino group, a nitro group, a hydroxyl group, a C₁-C₅ alkyl group, a C<3 branched alkyl group, a C₃-C₅ cycloalkoxyl group, an alkylamino group, a hydroxylhalide group, a C₁-C₅ alkylhalide group, a C<3 branched alkylhalide group, and a C₃-C₅ cycloalkoxylhalide group.

In accordance with another aspect of the present invention, a pharmaceutical composition including a 2,7-disubstituted anthraquinone derivative as claimed in claim 1 is provided.

In accordance with another aspect of the present invention, a pharmaceutical composition according to claim 11 is provided. The pharmaceutical composition is used for treating a cancer and further includes an additive selected from a group consisting of a pharmaceutically acceptable carrier, a dilutent, an excipient and a combination thereof.

Preferably, the 2,7-disubstituted anthraquinone derivative has an effective dose.

In accordance with another aspect of the present invention, a pharmaceutical composition according to claim 10 is provided. The pharmaceutical composition is used for inhibiting telomerase of a cell and including an additive selected from a group consisting of a pharmaceutically acceptable carrier, a dilutent, an excipient and a combination thereof.

Preferably, the cell is a mammalian cell.

In accordance with another aspect of the present invention, a preparation method of a 2,7-disubstituted anthraquinone derivative is provided. The preparation method includes steps of: (a) providing 2,7-diaminoanthraquinone; and (b) acetylating 2,7-diaminoanthraquinone to be bounded with a first side chain having chloride.

Preferably, 2,7-diaminoanthraquinone is obtained by steps of: (a) oxidizing anthrone to generate a first compound; (b) nitrifying the first compound to generate a second compound; and (c) reducing the second compound by sodium sulfide.

Preferably, the step (b) further includes steps of: (b1) dissolving 2,7-diaminoanthraquinone with N,N-dimethylformamide; (b2) catalyzing 2,7-diaminoanthraquinone with pyridine under an ice bath; (b3) causing 2,7-diaminoanthraquinone to react with nitrogen gas; and (b4) stirring 2,7-diaminoanthraquinone at room temperature for 24 hours in the dark.

Preferably, the preparation method further includes a step of: (c) after the step (a), aminating the 2,7-disubstituted anthraquinone derivative to be bounded with a second side chain having an amino group.

Preferably, the step (c) is reacted at a closed device, at reaction temperature of 130 to 150° C., under an oil bath and for a reaction time of 30 to 50 minutes.

The compounds of the present invention supplement with various excipients, carriers or diluents if necessary. For instance, the binders, such as starch and sodium carboxymethylcellulose (Na-CMC), etc., are added therein to prepare as particles and tablets, or fill as capsules in accordance with the prior preparation method. In addition, the pH value is adjusted by phosphate-buffered saline (PBS), so as to achieve the appropriate pH to prepare as the injection forms. Further, the permeated improver is added to prepare as the skin-adsorbed forms.

In accordance with the above-mentioned compounds and the preparation method thereof, the present invention not only provides the compounds for achieving the arrest of continuously-mitotic cancer cells by inhibiting the telomerase activity of cancer cells, but also provides the related synthetic method for simply and easily synthesizing the compounds of the present invention under the best conditions and the best molar ratio the reaction utilized.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

The anthraquinone compounds disclosed in the present invention mainly are 2,7-diaminoanthraquinone and its derivatives, and the preferred embodiments of the synthetic method thereof are described as follows.

### I. Synthesis of 2,7-diaminoanthraquinone

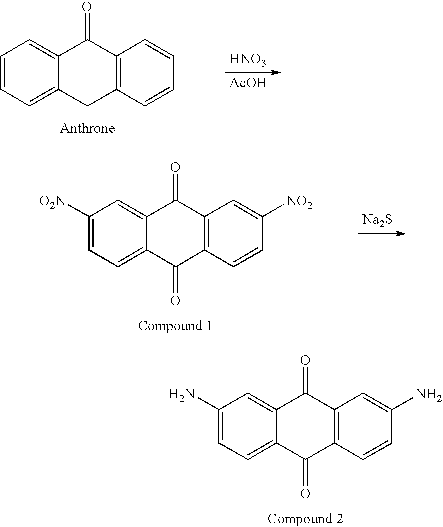

In the present invention, anthrone is firstly oxidized and nitrified, and the nitro group (—NO₂) of the aforementioned compound is reduced as amino group (—NH₂) with sodium sulfide. The obtained compound then is being the starting material of the series synthetic derivatives of the present invention. The detailed steps are described as follows.

Anthrone of 0.01 mol (1.942 g) was added and mixed with 12 ml of fuming nitric acid under the ice bath, and reacted for 1 to 2 hours. The reacted mixture was added and mixed with 35 ml of glacial acetic acid under the ice bath, and stirred for 10 to 20 minutes until the appearance of the precipitate. The precipitate was obtained by filtration, and the light-yellow crystalline solid (Compound 1) was obtained by re-crystallizing with glacial acetic acid. Compound 1 of 0.005 mol (1.49 g) was mixed with 56 ml of ethanol to be a suspension. This suspension was further added and reacted with the reducing reagent, which was prepared by dissolving 53.5 millimole (mmol) (2.14 g) of sodium hydroxide into 95 ml of water and then adding in 22.5 mmol (5.4 g) of sodium sulfide nonahydrate. The mixture was heated to circulate for 6 hours, then the mixture was disposed overnight at room temperature. The precipitate was obtained by filtering, washing with water repeatedly and drying. The orange-red solid (Compound 2) was obtained by re-crystallizing the obtained precipitate with ethanol.

### II. Synthesis of 2,7-disubstituted Anthraquinone Derivatives

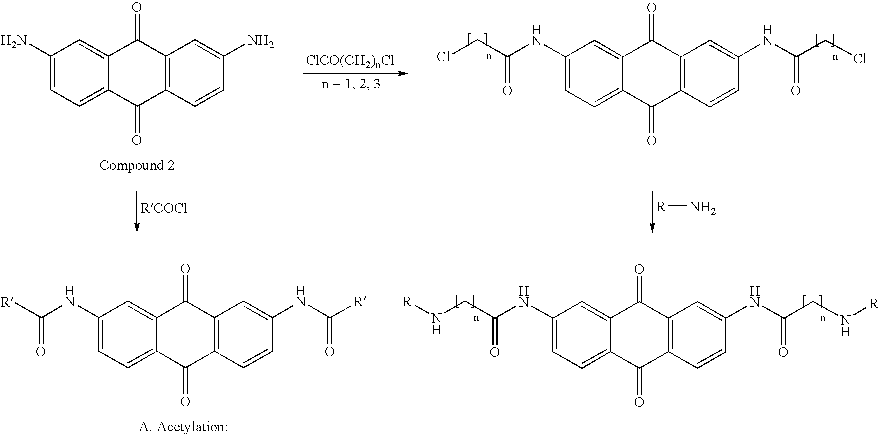

Compound 2 of 1 mmol (0.238 g) was dissolved in 20 ml of N,N-dimethylformamide, and 0.5 ml of pyridine was added to catalyze Compound 2 under the ice bath. Chloroacetyl chloride of 6 mmol (0.5 ml) then was added, the ice bath was removed, nitrogen gas was injected, and the stirring reaction was performed at room temperature for 1 day. The reacted mixture was added into a small amount of crushed ice, and the precipitate appeared now. The precipitate was obtained by filtration, and washed with diethyl ether. Finally, Compound 3 was obtained by re-crystallizing the precipitate with ethanol. Compounds 3 to 34 could be obtained by this method.

B. Amination:

Compound 3 of 1 mmol (0.391 g) was dissolved in 20 ml of N,N-dimethylformamide, and 10 mmol (1 ml) of diethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 4 was obtained by re-crystallizing the precipitate with ethanol. Compounds 4 to 14, and 16 to 24 could be obtained by the same method.

Thirty four (34) anthraquinone compounds disclosed in the present invention were obtained by the abovementioned synthetic methods. The physical and chemical characteristics thereof were further determined, wherein the melting point (mp) was determined by the melting point apparatus (Melting Point B-545, Büchi), and the determined result was counted to the first decimal place. For the consistence, the unit's place is adapted and the decimal point was rounded off. Infrared (IR, KBr) was determined by the Perkin-Elmer 983G spectrometer. Mass spectrometry (MS) was determined in Germany and in National Chiao Tung University, Taiwan respectively. The ¹H-NMR and ¹³C-NMR were determined by the Varian Gemini-300 (300 MHz). The determined physical and chemical characteristics of each compound were described respectively as follows.

### (1). 2,7-dinitroanthraquinone (Compound 1)

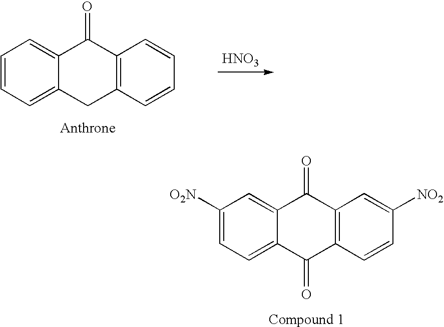

Anthrone of 0.01 mol (1.942 g) was added and mixed with 12 ml of fuming metric acid under the ice bath and reacted for 1 to 2 hour. The reacted mixture was added into 35 ml of glacial acetic acid under the ice bath and stirred for 10 to 20 minutes so as to appear the precipitate. The precipitate was purified by filtration. The light-yellow solid (compound 1) was obtained by re-crystallizing with glacial acetic acid.

Compound 1 has the following properties: yield: 39%; mp: 284° C. (AcOH) (lit.³² mp: 290-291° C.); IR (KBr) (cm⁻¹): 1303, 1542 (NO), 1677 (CO); MS (EI, 70 ev)=298.0 (M⁺); ¹H-NMR (300 MHz, DMSO) δ (ppm): 8.47 (d, J=8.1 Hz, 2H, H-4,5), 8.70 (dd, J=8.4, 2.4 Hz, 2H, H-3,6), 8.83 (d, J=2.1 Hz, 2H, H-1,8); ¹³C-NMR (300 MHz, DMSO) δ (ppm): 120.88 (C-3,6), 128.09 (C-1,8), 128.61 (C-4,5), 133.75 (C-8a,9a), 136.33 (C-4a,5a), 150.34 (C-2,7), 179.19 (CO), 179.78 (CO).

### (2). 2,7-Diaminoanthraquinone (Compound 2)

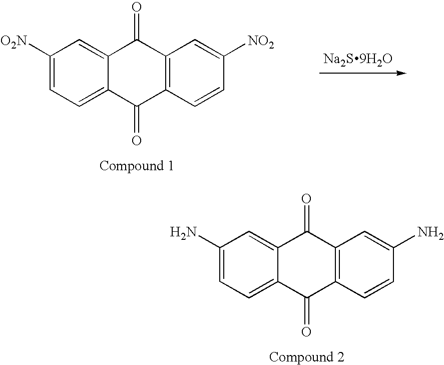

Compound 1 of 0.005 mol (1.49 g) was mixed with 56 ml of ethanol for being a suspension. This suspension then was added and reacted with the reducing agent, which was prepared by dissolving 53.5 mmol (2.14 g) of sodium hydroxide into 95 ml of water and then adding in 22.5 mmol (5.4 g) of sodium sulfide nonahydrate. The mixture was heated to circulate for 6 hours, then the mixture was placed overnight at room temperature. The precipitate was purified by filtrating, washing with water repeatedly and drying. The orange-red solid (Compound 2) was obtained by re-crystallizing the obtained precipitate with ethanol.

Compound 2 has the following properties: yield: 77%; mp: 342° C. (EtOH) (lit.³² mp: 337-338° C.); IR (KBr) (cm⁻¹): 1641, 1672 (CO), 3326, 3399 (NH₂); MS (EI, 70 ev)=238.1 (M⁺); ¹H-NMR (300 MHz, DMSO) δ (ppm): 6.39 (s, 4H, NH₂), 6.92 (dd, J=8.7, 2.4 Hz, 2H, H-3,6), 7.27 (d, J=2.1 Hz, 2H, H-1,8), 7.87 (d, J=8.7 Hz, 2H, H-4,5); ¹³C-NMR (300 MHz, DMSO) δ (ppm): 109.24 (C-1,8), 117.53 (C-3,6), 121.61 (C-4a,5a), 128.43 (C-4,5), 134.42 (C-8a,9a), 153.12 (C-2,7), 178.87 (CO), 183.76 (CO).

### (3). 2,7-bis(chloroacetamido)anthraquinone (Compound 3)

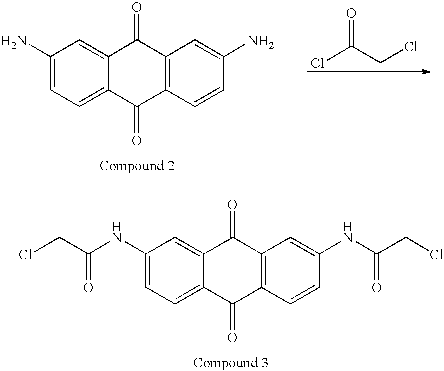

Compound 2 of 1 mmol (0.238 g) was dissolved in 20 ml of N,N-dimethylformamide, and 0.5 ml of pyridine was added to catalyze Compound 2 under the ice bath. Chloroacetyl chloride of 6 mmol (0.5 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 3 was obtained by re-crystallizing the precipitate with ethanol.

Compound 3 has the following properties: yield: 59%; mp: 286° C. (EtOH); IR (KBr) (cm⁻¹): 1672 (CO), 1718 (CONH), 3315 (NH); MS (EI, 70 ev)=390.0 (M⁺); ¹H-NMR (300 MHz, DMSO) δ (ppm): 4.32 (s, 4H, CH₂), 7.97 (dd, J=8.4, 2.1 Hz, 2H, H-3,6), 8.06 (d, J=8.4 Hz, 2H, H-4,5), 8.33 (d, J=2.1 Hz, 2H, H-1,8), 10.86 (s, 2H, NH); ¹³C-NMR (300 MHz, DMSO) δ (ppm): 42.99 (COCH₂), 115.61 (C-1,8), 123.50 (C-3,6), 127.87 (C-4a,5a), 127.97 (C-4,5), 133.63 (C-4a,9a), 143.21 (C-2,7), 165.08 (NCO), 179.79 (CO), 181.67 (CO).

### (4). 2,7-Bis[2-(diethylamino)acetamido]anthraquinone (Compound 4)

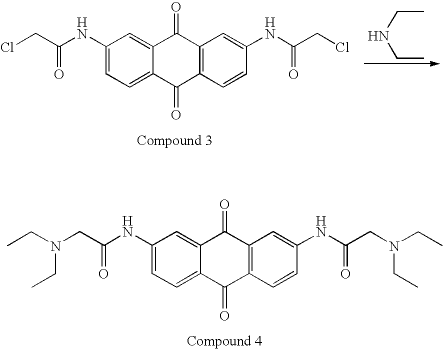

Compound 3 of 1 mmol (0.391 g) was dissolved in 20 ml of N,N-dimethylformamide, and 10 mmol (1 ml) of diethylamine was added. The mixture then was catalyzed with 0.5 ml of pyridine. This mixture was reacted in a close device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 4 was obtained by re-crystallizing the precipitate with ethanol.

Compound 4 has the following properties: yield: 61%; mp: 241° C. (EtOH); IR (KBr) (cm⁻¹): 1679 (CO), 1702 (CONH), 3250 (NH); MS (EI, 70 ev)=464.2 (M⁺); ¹H-NMR (300 MHz, DMSO) δ (ppm): 1.01 (t, J=7.0 Hz, 12H, CH₃), 2.61 (q, J=6.9 Hz, 8H, NCH₂), 3.22 (s, COCH₂), 8.09 (d, J=8.4 Hz, 2H, H-4,5), 8.13 (dd, J=9.6, 1.2 Hz, 2H, H-3,6), 8.50 (s, 2H, H-1,8), 10.27 (s, 2H, NH); ¹³C-NMR (300 MHz, DMSO) δ (ppm): 11.30 [N(CH₂CH₃)₂], 47.17 [N(CH₂CH₃)₂], 56.80 (COCH₂), 115.73 (C-1,8), 123.56 (C-3,6), 127.57 (C-4a,5a), 127.62 (C-4,5), 133.58 (C-4a,9a), 143.15 (C-2,7), 170.41 (NCO), 179.75 (CO), 181.80 (CO).

### (5). 2,7-Bis[2-(butylamino)acetamido]anthraquinone (Compound 5)

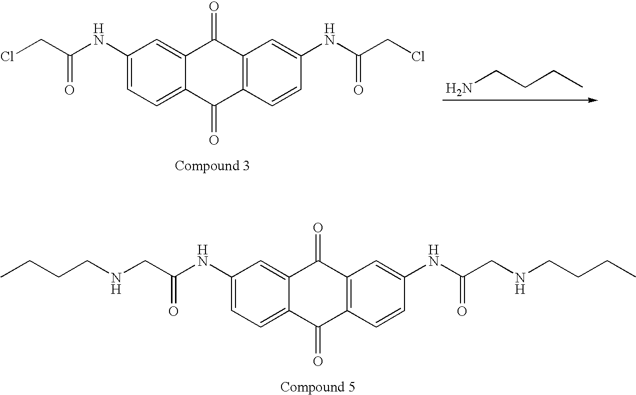

Compound 3 of 1 mmol (0.391 g) was dissolved in 20 ml of N,N-dimethylformamide, and 10 mmol (1 ml) of butylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 5 was obtained by re-crystallizing the precipitate with ethanol.

Compound 5 has the following properties: yield: 40%; mp: 125° C. (EtOH); IR (KBr) (cm⁻¹): 1671 (CO), 3331 (NH); MS (EI, 70 ev)=464.3 (M⁺); ¹H-NMR (300 MHz, CDCl₃) δ (ppm): 0.94 (t, J=7.2 Hz, 6H, CH₃), 1.41 (m, 4H, CH₂CH₃), 1.52 (m, 4H, CH₂CH₂CH₂), 2.69 (t, J=6.8 Hz, 4H, NCH₂), 3.41 (s, 4H, COCH₂), 8.09 (d, J=1.5 Hz, 2H, H-1,8), 8.25 (d, J=8.4 Hz, 2H, H-4,5), 8.31 (dd, J=8.4, 2.1 Hz, 2H, H-3,6), 9.85 (s, 2H, NH); ¹³C-NMR (300 MHz, CDCl₃) δ (ppm): 13.82 (CH₃), 20.23 (NCH₂CH₂CH₂), 32.15 (NCH₂CH₂), 50.04 (NCH₂), 53.03 (COCH₂), 116.29 (C-1,8), 123.88 (C-3,6), 128.96 (C-4a,5a), 129.14 (C-4,5), 34.53 (C-4a,9a), 142.79 (C-2,7), 170.73 (NCO), 180.72 (CO), 182.45 (CO).

### (6). 2,7-Bis[2-(pyrrolidino)acetamido]anthraquinone (Compound 6)

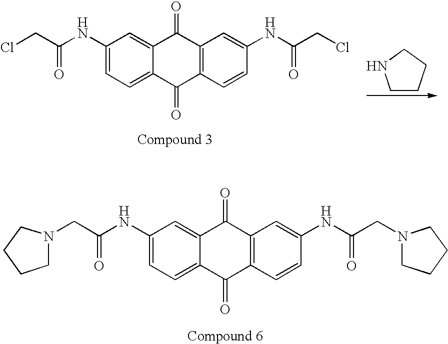

Compound 3 of 1 mmol (0.391 g) was dissolved in 20 ml of N,N-dimethylformamide, and 10 mmol (0.9 ml) of pyrrolidine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 6 was obtained by re-crystallizing the precipitate with ethanol.

Compound 6 has the following properties: yield: 33%; mp: 201° C. (EtOH); IR (KBr) (cm⁻¹): 1674 (CO), 1699 (CONH), 3358 (NH); MS (EI, 70 ev)=460.2 (M⁺); ¹H-NMR (300 MHz, CDCl₃) δ (ppm): 1.88 (m, 8H, H-3′,4′), 2.71 (t, 8H, H-2′,5′), 3.32 (s, 4H, COCH₂), 8.09 (d, J=2.1 Hz, 2H, H-1,8), 8.28 (d, J=8.4 Hz, 2H, H-4,5), 8.37 (dd, J=8.7, 2.4 Hz, 2H, H-3,6), 9.58 (s, 2H, NH); ¹³C-NMR (300 MHz, CDCl₃) δ (ppm): 24.11 (C-3′,4′), 54.65 (COCH₂), 59.79 (C-2′,5′), 116.40 (C-1,8), 124.07 (C-3,6), 129.00 (C-4a,5a), 129.17 (C-4,5), 134.50 (C-4a,9a), 142.88 (C-2,7), 169.68 (NCO), 180.71 (CO), 182.60 (CO).

### (7). 2,7-Bis[2-(piperidino)acetamido]anthraquinone (Compound 7)

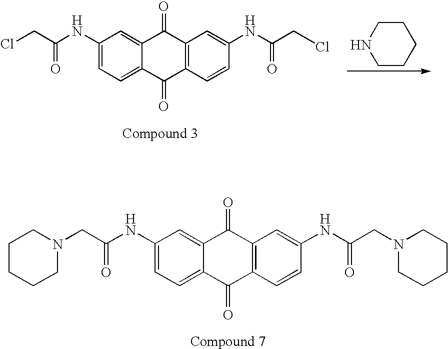

Compound 3 of 1 mmol (0.391 g) was dissolved in 20 ml of N,N-dimethylformamide, and 10 mmol (1 ml) of piperidine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 7 was obtained by re-crystallizing the precipitate with ethanol.

Compound 7 has the following properties: yield: 51%; mp: 243° C. (EtOH); IR (KBr) (cm⁻¹): 1678 (CO), 1706 (CONH), 3242 (NH); MS (EI, 70 ev)=488.3 (M⁺); ¹H-NMR (300 MHz, CDCl₃) δ (ppm): 1.49 (m, 4H, H-4′), 1.65 (m, 8H, H-3′,5′), 2.54 (t, J=4.8 Hz, 8H, H-2′,6′), 3.09 (s, 4H, COCH₂), 8.07 (d, J=2.1 Hz, 2H, H-1,8), 8.23 (d, J=8.4 Hz, 2H, H-4,5), 8.28 (dd, J=8.7, 1.8 Hz, 2H, H-3,6), 9.68 (s, 2H, NH); ¹³C-NMR (300 MHz, CDCl₃) δ (ppm): 23.52 (C-3′,5′), 26.20 (C-4′), 54.89 (C-2′,6′), 62.77 (COCH₂), 116.40 (C-1,8), 124.11 (C-3,6), 129.12 (C-4a,5a), 129.25 (C-4,5), 134.62 (C-4a,9a), 142.87 (C-2,7), 169.58 (NCO), 180.83 (CO), 182.67 (CO).

### (8). 2,7-Bis[2-(dimethylamino)acetamido]anthraquinone (Compound 8)

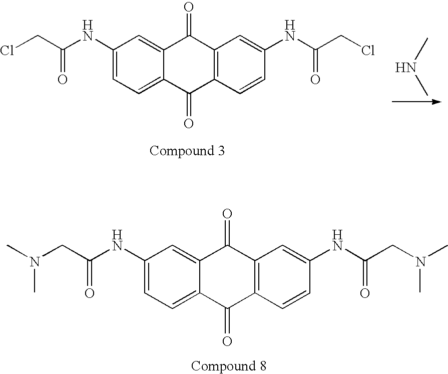

Compound 3 of 1 mmol (0.391 g) was dissolved in 20 ml of N,N-dimethylformamide, and 10 mmol (1.3 ml) of dimethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 8 was obtained by re-crystallizing the precipitate with ethanol.

Compound 8 has the following properties: yield: 26%; mp: 218° C. (EtOH); IR (KBr) (cm⁻¹): 1630, 1672 (CO), 1701 (CONH), 3330 (NH); MS (EI, 70 ev)=408.1 (M⁺); ¹H-NMR (300 MHz, CDCl₃) δ (ppm): 2.38 (s, 12H, CH₃), 3.09 (s, 4H, CH₂), 8.04 (d, J=2.1 Hz, 2H, H-1,8), 8.19 (d, J=8.4 Hz, 2H, H-4,5), 8.28 (dd, J=8.4, 2.1 Hz, 2H, H-3,6), 9.55 (s, 2H, NH); ¹³C-NMR (300 MHz, CDCl₃) δ (ppm): 45.98 [N(CH₃)₂], 63.56 (COCH₂), 116.39 (C-1,8), 123.99 (C-3,6), 129.05 (C-4a,5a), 129.20 (C-4,5), 134.54 (C-4a,9a), 142.84 (C-2,7), 169.32 (NCO), 180.74 (CO), 182.50 (CO).

### (9). 2,7-Bis[2-(propylamino)acetamido]anthraquinone (Compound 9)

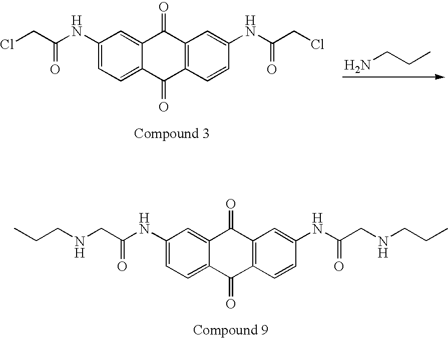

Compound 3 of 1 mmol (0.391 g) was dissolved in 20 ml of N,N-dimethylformamide, and 10 mmol (0.9 ml) of propylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 9 was obtained by re-crystallizing the precipitate with ethanol.

Compound 9 has the following properties: yield: 30%; mp: 143° C. (EtOH); IR (KBr) (cm⁻¹): 1670 (CO), 3333 (NH); MS (EI, 70 ev)=436.2 (M⁺); ¹H-NMR (300 M Hz, CDCl₃) δ (ppm): 0.98 (t, J=7.5 Hz, 6H, CH₃), 1.56 (m, 4H, CH₂), 2.66 (t, J=6.9 Hz, 4H, NCH₂), 3.41 (s, 4H, COCH₂), 8.10 (d, J=1.8 Hz, 2H, H-1,8), 8.25 (d, J=8.1 Hz, 2H, H-4,5), 8.30 (dd, J=8.4, 1.8 Hz, 2H, H-3,6), 9.84 (s, 2H, NHCO); ¹³C-NMR (300 MHz, DMSO) δ (ppm): 11.17 (CH₃), 22.04 (CH₂CH₂), 50.51 (CH₂CH₂), 52.35 (COCH₂), 115.45 (C-1,8), 123.36 (C-3,6), 127.62 (C-4a,5a), 127.83 (C-4,5), 133.71 (C-4a,9a), 143.50 (C-2,7), 171.08 (NCO), 179.90 (CO), 181.98 (CO).

### (10). 2,7-Bis[2-(ethylamino)acetamido]anthraquinone (Compound 10)

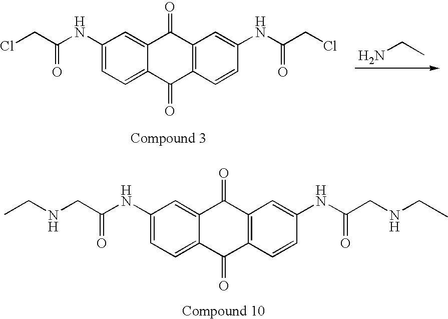

Compound 3 of 1 mmol (0.391 g) was dissolved in 20 ml of N,N-dimethylformamide, and 10 mmol (0.6 ml) of ethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 10 was obtained by re-crystallizing the precipitate with ethanol.

Compound 10 has the following properties: yield: 55%; mp: 145° C. (EtOH); IR (KBr) (cm⁻¹): 1670 (CO), 3330 (NH); MS (EI, 70 ev)=408 (M⁺); ¹H-NMR (300 MHz, CDCl₃) δ (ppm): 1.18 (t, J=7.2 Hz, 6H, CH₃), 2.75 (q, J=7.2 Hz, 4H, NCH₂), 3.42 (s, 4H, COCH₂), 8.12 (d, J=1.8 Hz, 2H, H-1,8), 8.28 (d, J=8.4 Hz, 2H, H-4,5), 8.35 (dd, J=8.7, 2.1 Hz, 2H, H-3,6), 9.85 (s, 2H, NHCO); ¹³C-NMR (300 MHz, DMSO) δ (ppm): 14.39 (CH₃), 42.78 (CH₂CH₃), 52.18 (COCH₂), 115.50 (C-1,8), 123.39 (C-3,6), 127.62 (C-4a,5a), 127.77 (C-4,5), 133.69 (C-4a,9a), 143.48 (C-2,7), 171.07 (NCO), 179.89 (CO), 181.96 (CO).

### (11). 2,7-Bis[2-(N-methylpiperazino)acetamido]anthraquinone (Compound 11)

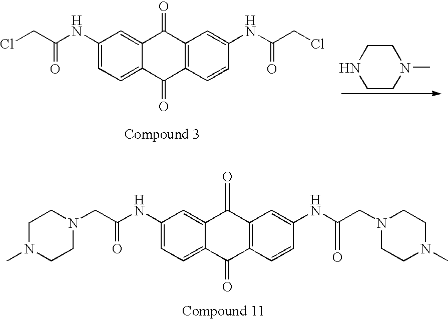

Compound 3 of 1 mmol (0.391 g) was dissolved in 20 ml of N,N-dimethylformamide, and 10 mmol (1.1 ml) of N-methylpiperazine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 11 was obtained by re-crystallizing the precipitate with ethanol.

Compound 11 has the following properties: yield: 46%; mp: 173° C. (EtOH); IR (KBr) (cm⁻¹): 1672, 1691 (CO), 1710 (CONH), 3241 (NH); MS (EI, 70 ev)=518 (M⁺); ¹H-NMR (300 MHz, CDCl₃) δ (ppm): 2.35 (s, 6H, CH₃), 2.55 (s, 8H, H-3′,5′), 2.68 (s, 8H, H-2′,6′), 3.19 (s, 4H, COCH₂), 8.12 (d, J=1.5 Hz, 2H, H-1,8), 8.28 (d, J=8.7 Hz, 2H, H-4,5), 8.32 (dd, J=8.7, 1.8 Hz, 2H, H-3,6), 9.55 (s, 2H, NH); ¹³C-NMR (300 MHz, DMSO) δ (ppm): 45.27 (CH3), 52.17 (C-2′,6′), 54.02 (C-3′,5′), 61.35 (COCH₂), 115.83 (C-1,8), 123.67 (C-3,6), 127.77 (C-4a,5a), 127.82 (C-4,5), 133.73 (C-4a,9a), 143.51 (C-2,7), 169.05 (NCO), 180.01 (CO), 182.06 (CO).

### (12) 2,7-Bis[2-(piperazino)acetamido]anthraquinone (Compound 12)

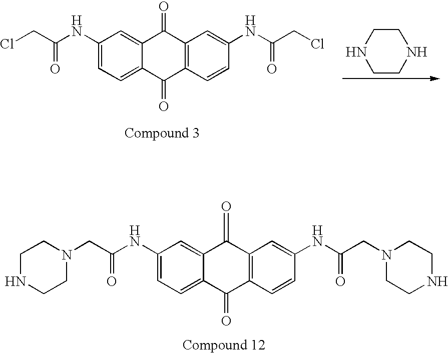

Compound 3 of 1 mmol (0.391 g) was dissolved in 20 ml of N,N-dimethylformamide, and 10 mmol (0.9 ml) of piperazine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 12 was obtained by re-crystallizing the precipitate with ethanol.

Compound 12 has the following properties: yield: 19%; mp: 173° C. (EtOH); IR (KBr) (cm⁻¹): 1643, 1668 (CO), 1699 (CONH), 3332 (NH); MS (EI, 70 ev)=490 (M⁺); ¹H-NMR (300 MHz, CDCl₃) δ (ppm): 2.60 (s, 8H, H-3′,5′), 3.00 (t, J=4.8 Hz, 8H, H-2′,6′), 3.16 (s, 4H, COCH₂), 8.11 (d, J=1.8 Hz, 2H, H-1,8), 8.28 (d, J=8.7 Hz, 2H, H-4,5), 8.33 (dd, J=8.7, 2.1 Hz, 2H, H-3,6), 9.58 (s, 2H, NH); ¹³C-NMR (300 MHz, DMSO) δ (ppm): 44.81 (C-3′,5′), 53.51 (C-2′,6′), 62.07 (COCH₂), 115.85 (C-1,8), 123.71 (C-3,6), 127.73 (C-4a,5a), 127.77 (C-4,5), 133.71 (C-4a,9a), 143.43 (C-2,7), 169.09 (NCO), 179.98 (CO), 182.02 (CO).

### (13). 2,7-Bis[2-(isopropylamino)acetamido]anthraquinone (Compound 13)

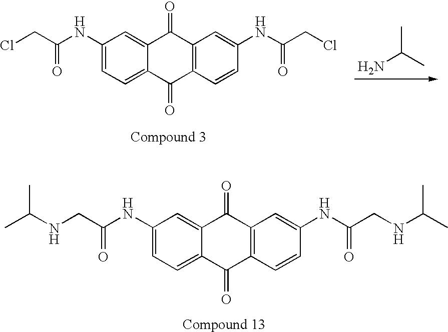

Compound 3 of 1 mmol (0.391 g) was dissolved in 20 ml of N,N-dimethylformamide, and 10 mmol (0.9 ml) of isopropylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 13 was obtained by re-crystallizing the precipitate with ethanol.

Compound 13 has the following properties: yield: 44%; mp: 158° C. (EtOH); IR (KBr) (cm⁻¹): 1672 (CO), 3323 (NH); MS (EI, 70 ev)=436 (M⁺); ¹H-NMR (300 MHz, DMSO) δ (ppm): 1.01 (d, J=6.0 Hz, 12H, CH₃), 2.76 (m, 2H, CH), 3.35 (s, 4H, COCH₂), 8.07 (dd, J=8.4, 2.1 Hz, 2H, H-3,6), 8.14 (d, J=8.7 Hz, 2H, H-4,5), 8.49 (d, J=1.8 Hz, 2H, H-1,8); ¹³C-NMR (300 MHz, DMSO) δ (ppm): 21.99 [CH(CH₃)₂], 47.65 (CH), 50.06 (COCH₂), 115.45 (C-1,8), 123.36 (C-3,6), 127.64 (C-4a,5a), 127.75 (C-4,5), 133.70 (C-8a,9a), 143.37 (C-2,7), 171.20 (NCO), 179.86 (CO), 181.93 (CO).

### (14). 2,7-Bis[2-(isobutylamino)acetamido]anthraquinone (Compound 14)

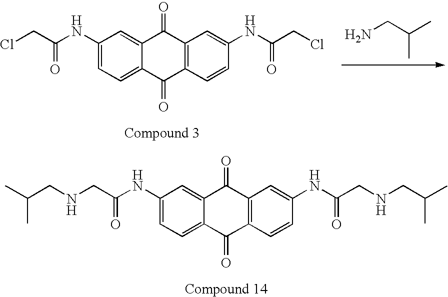

Compound 3 of 1 mmol (0.391 g) was dissolved in 20 ml of N,N-dimethylformamide, and 10 mmol (1 ml) of isobutylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 14 was obtained by re-crystallizing the precipitate with ethanol.

Compound 14 has the following properties: yield: 73%; mp: 146° C. (EtOH); IR (KBr) (cm⁻¹): 1670 (CO), 3335 (NH); MS (EI, 70 ev): m/z (%)=465 (M⁺); ¹H-NMR (300 MHz, CDCl₃) δ (ppm): 0.99 (d, J=6.6 Hz, 12H, CH₃), 1.79 (m, 2H, CH), 2.50 (d, J=6.3 Hz, 4H, CH₂), 3.41 (s, 4H, COCH₂), 8.13 (s, 2H, H-1,8), 8.28 (s, 4H, H-3,4,5,6), 9.86 (s, 2H, NHCO); ¹³C-NMR (300 MHz, DMSO) δ (ppm): 20.06 [CH(CH₃)₂], 27.49 (CH), 52.60 (COCH₂), 56.74 (CH₂), 115.38 (C-1,8), 123.29 (C-3,6), 127.61 (C-4a,5a), 127.85 (C-4,5), 133.71 (C-4a,9a), 143.47 (C-2,7), 171.13 (NCO), 179.86 (CO), 181.95 (CO).

### (15). 2,7-Bis(3-chloropropionamido)anthraquinone (Compound 15)

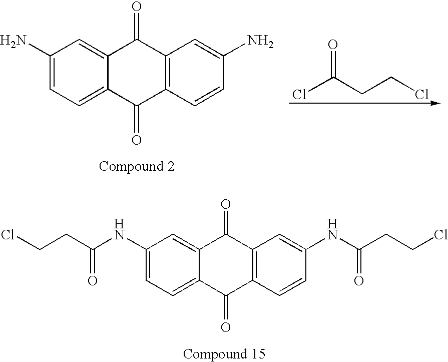

Compound 2 of 1 mmol (0.238 g) was dissolved in 20 ml of N,N-dimethylformamide, and 0.5 ml of pyridine was added to catalyze Compound 2 under the ice bath. 3-Chloropropioyl chloride of 6 mmol (0.5 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was purified by filtration, and washed with diethyl ether. Finally, Compound 15 was obtained by re-crystallizing the precipitate with ethanol.

Compound 15 has the following properties: yield: 58%; mp: 281° C. (EtOH); IR (KBr) (cm⁻¹): 1672 (CO), 1702 (CONH), 3333 (NH); MS (EI, 70 ev)=418.0 (M⁺); ¹H-NMR (300 MHz, DMSO) δ (ppm): 2.91 (t, J=5.9 Hz, 4H, COCH₂), 3.90 (t, J=5.9 Hz, 4H, CH₂Cl), 8.01 (d, J=8.7 Hz, 2H, H-3,6), 8.09 (d, J=8.7 Hz, 2H, H-4,5), 8.40 (s, 2H, H-1,8), 10.72 (s, 2H, NH); ¹³C-NMR (300 MHz, DMSO) δ (ppm): 38.86 (COCH₂), 39.82 (CH₂Cl), 115.31 (C-1,8), 123.14 (C-3,6), 127.54 (C-4a,5a), 127.67 (C-4,5), 133.53 (C-4a,9a), 143.44 (C-2,7), 168.33 (NCO), 179.63 (CO), 181.66 (CO).

### (16). 2,7-Bis[3-(diethylamino)propionamido]anthraquinone (Compound 16)

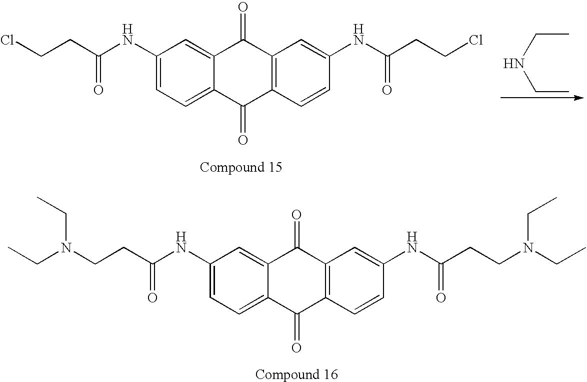

Compound 15 of 1 mmol (0.419 g) was dissolved in 20 ml of N,N-dimethylformamide, and 10 mmol (1 ml) of diethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 16 was obtained by re-crystallizing the precipitate with ethanol.

Compound 16 has the following properties: yield: 55%; mp: 193° C. (EtOH) (lit.³² mp: 215° C.); IR (KBr) (cm⁻¹): 1672 (CO), 1697 (CONH), 3323 (NH); MS (EI, 70 ev)=492.3 (M⁺); ¹H-NMR (300 MHz, CDCl₃) δ (ppm): 1.15 (t, J=7.2 Hz, 12H, CH₃), 2.54 (t, J=5.7 Hz, 4H, CH₂N), 2.70 (q, J=7.2 Hz, 8H, NCH₂), 2.79 (t, J=5.6 Hz, 4H, COCH₂), 8.02 (s, 2H, H-1,8), 8.23 (s, 4H, H-3,4,5,6), 12.01 (s, 2H, NH); ¹³C-NMR (300 MHz, CDCl₃) δ (ppm): 11.44 [N(CH₂CH₃)₂], 33.27 (COCH₂), 46.12 [N(CH₂CH₃)₂], 48.79 (CH₂N), 116.35 (C-1,8), 124.37 (C-3,6), 128.99 (C-4a,5a), 129.11 (C-4,5), 134.78 (C-8a,9a), 144.04 (C-2,7), 171.32 (NCO), 181.06 (CO), 182.85 (CO).

### (17). 2,7-Bis[3-(butylamino)propionamido]anthraquinone (Compound 17)

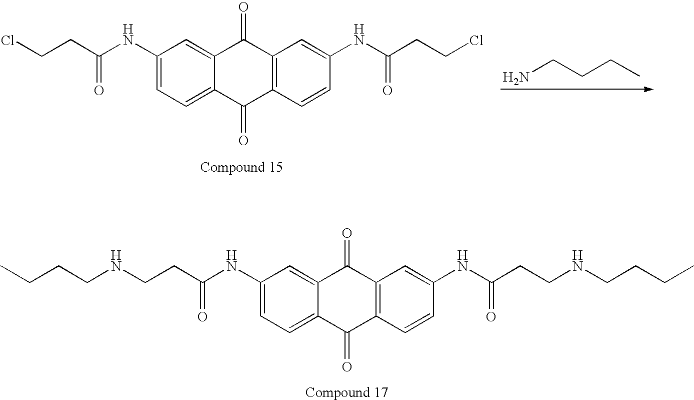

Compound 15 of 1 mmol (0.419 g) was dissolved in 20 ml of N,N-dimethylformamide, and 10 mmol (1 ml) of butylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 17 was obtained by re-crystallizing the precipitate with ethanol.

Compound 17 has the following properties: yield: 22%; mp: 168° C. (EtOH); IR (KBr) (cm⁻¹): 1648, 1671 (CO), 3333 (NH); ¹H-NMR (300 MHz, CDCl₃) δ (ppm): 2.41 (s, 18H, CH₂CH₂CH₂CH₃), 2.53 (t, J=5.4 Hz, 4H, CH₂N), 2.67 (t, J=5.4 Hz, 4H, COCH₂), 8.01 (d, J=1.5 Hz, 2H, H-1,8), 8.23 (d, J=3.0 Hz, 4H, H-3,4,5,6), 11.68 (s, 2H, CONH); ¹³C-NMR (300 MHz, CDCl₃) δ (ppm): 13.30 (CH₃), 19.38 (NCH₂CH₂CH₂), 30.82 (NCH₂CH₂), 34.37 (COCH₂), 45.04 (NCH₂), 46.43 (CH₂N), 115.33 (C-1,8), 123.23 (C-3,6), 127.50 (C-4a,5a), 127.83 (C-4,5), 133.73 (C-4a,9a), 143.38 (C-2,7), 170.04 (NCO), 179.36 (CO), 182.09 (CO).

### (18). 2,7-Bis[3-(pyrrolidino)propionamido]anthraquinone (Compound 18)

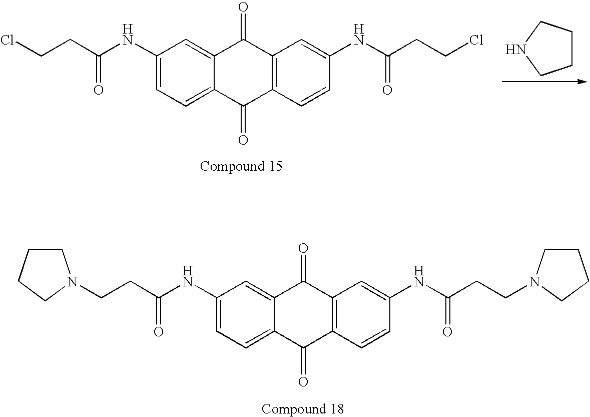

Compound 15 of 1 mmol (0.419 g) was dissolved in 20 ml of N,N-dimethylformamide, and 10 mmol (0.8 ml) of pyrrolidine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 18 was obtained by re-crystallizing the precipitate with ethanol.

Compound 18 has the following properties: yield: 49%; mp: 233° C. (EtOH) (lit.³² mp: 232° C.); IR (KBr) (cm⁻¹): 1645, 1672 (CO), 1698 (CONH), 3331 (NH); MS (EI, 70 ev)=488 (M⁺); ¹H-NMR (300 MHz, CDCl₃) δ (ppm): 1.95 (s, 8H, H-3′,4′), 2.57 (t, J=6.0 Hz, 4H, CH₂N), 2.71 (s, 8H, H-2′,5′), 2.87 (t, J=5.7 Hz, 4H, COCH₂), 7.98 (d, J=2.1 Hz, 2H, H-1,8), 8.17 (dd, J=8.4, 2.1 Hz, 2H, H-3,6), 8.24 (d, J=8.7 Hz, 2H, H-4,5), 11.98 (s, 2H, NH); ¹³C-NMR (300 MHz, CDCl₃) δ (ppm): 22.36 (C-3′,4′), 35.73 (COCH₂), 50.79 (CH₂N), 57.83 (C-2′,5′), 115.31 (C-1,8), 123.15 (C-3,6), 127.51 (C-4a,5a), 127.87 (C-4,5), 133.74 (C-4a,9a), 143.37 (C-2,7), 170.73 (NCO), 179.93 (CO), 182.00 (CO).

### (19). 2,7-Bis[3-(piperidino)propionamido]anthraquinone (Compound 19)

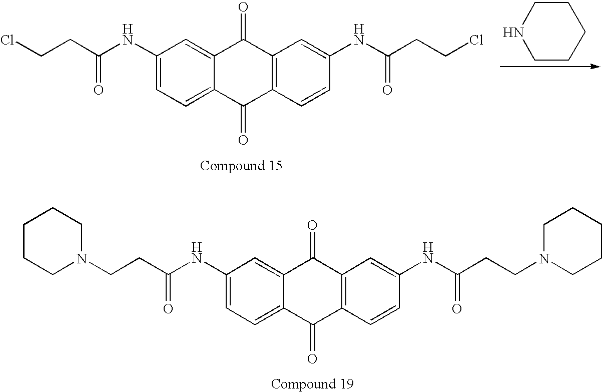

Compound 15 of 1 mmol (0.419 g) was dissolved in 20 ml of N,N-dimethylformamide, and 10 mmol (1 ml) of piperidine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 19 was obtained by re-crystallizing the precipitate with ethanol.

Compound 19 has the following properties: yield: 34%; mp: 266° C. (EtOH) (lit.³² mp: 240° C.); IR (KBr) (cm⁻¹): 1670 (CO), 1694 (CONH), 3315 (NH); MS (EI, 70 ev)=516 (M⁺); ¹H-NMR (300 MHz, CDCl₃) δ (ppm): 1.75 (t, J=5.1 Hz, 12H, H-3′,4′,5′,), 2.53-2.65 (m, 12H, CH₂N, H-2′,6′), 2.69 (t, J=4.8 Hz, 4H, COCH₂), 8.10 (d, J=1.5 Hz, 2H, H-1,8), 8.19 (dd, J=8.4, 2.4 Hz, 2H, H-3,6), 8.25 (d, J=8.7 Hz, 2H, H-4,5), 12.05 (s, 2H, NH); ¹³C-NMR (300 MHz, CDCl₃) δ (ppm): 24.09 (C-4′), 26.21 (C-3′,5′), 32.44 (COCH₂), 53.57 (CH₂N), 54.03 (C-2′,6′), 116.45 (C-1,8), 124.34 (C-3,6), 128.93 (C-4a,5a), 129.14 (C-4,5), 134.78 (C-4a,9a), 144.23 (C-2,7), 171.55 (NCO), 181.28 (CO), 182.91 (CO).

### (20). 2,7-Bis[3-(dimethylamino)propionamido]anthraquinone (Compound 20)

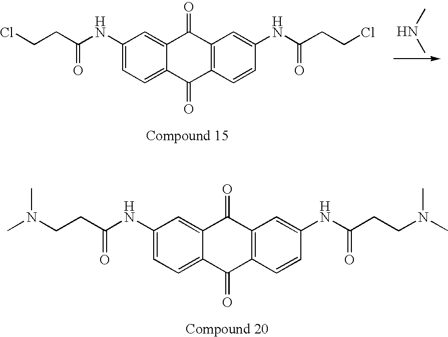

Compound 15 of 1 mmol (0.419 g) was dissolved in 20 ml of N,N-dimethylformamide, and 10 mmol (1.3 ml) of dimethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 20 was obtained by re-crystallizing the precipitate with ethanol.

Compound 20 has the following properties: yield: 20%; mp: 196° C. (EtOH) (lit.³² mp: 202-203° C.); IR (KBr) (cm⁻¹): 1651, 1673 (CO), 1698 (CONH), 3325 (NH); MS (EI, 70 ev)=436 (M⁺); ¹H-NMR (300 MHz, CDCl₃) δ (ppm): 2.4.1 (s, 12H, CH₃), 2.53 (t, J=5.4 Hz, 4H, CH₂N), 2.67 (t, J=5.4 Hz, 4H, COCH₂), 8.00 (s, 2H, H-1,8), 8.23 (s, 4H, H-3,4,5,6), 11.65 (s, 2H, NH); ¹³C-NMR (300 MHz, DMSO) δ (ppm): 33.40 (COCH₂), 44.38 [N(CH₃)₂], 54.89 (CH₂N), 116.87 (C-1,8), 124.70 (C-3,6), 129.08 (C-4,4a,5,5a), 134.71 (C-4a,9a), 144.04 (C-2,7), 171.49 (NCO), 179.99 (CO), 182.08 (CO).

### (21). 2,7-Bis[3-(propylamino)propionamido]anthraquinone (Compound 21)

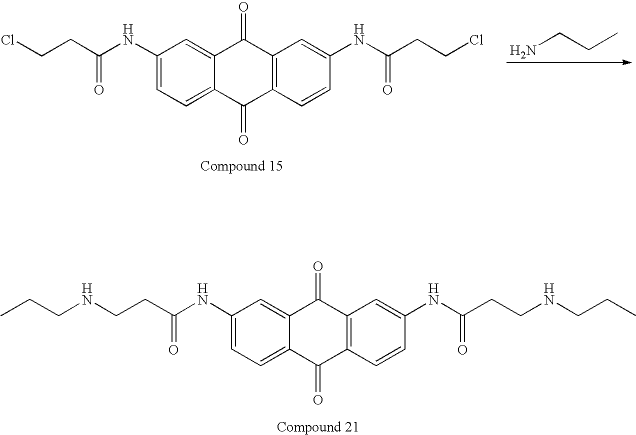

Compound 15 of 1 mmol (0.419 g) was dissolved in 20 ml of N,N-dimethylformamide, and 10 mmol (0.8 ml) of propylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 21 was obtained by re-crystallizing the precipitate with ethanol.

Compound 21 has the following properties: yield: 25%; mp: 170° C. (EtOH); IR (KBr) (cm⁻¹): 1651, 1671 (CO), 1698 (CONH), 3329 (NH); MS (EI, 70 ev)=462 (M⁺); ¹H-NMR (300 MHz, CDCl₃) δ (ppm): 2.41 (s, 14H, CH₂CH₂CH₃), 2.54 (t, J=6.0 Hz, 4H, CH₂N), 2.67 (t, J=6.0 Hz, 4H, COCH₂), 8.01 (s, 2H, H-1,8), 8.23 (d, J=1.8 Hz, 4H, H-3,4,5,6), 11.68 (s, 2H, NH); ¹³C-NMR (300 MHz, DMSO) δ (ppm): 29.17 (CH₃), 29.57 (NCH₂CH₂), 33.39 (COCH₂), 44.37 (CH₂N), 54.85 (NCH₂), 116.87 (C-1,8), 124.66 (C-3,6), 129.04 (C-4,4a,5,5a), 134.69 (C-4a,9a), 144.04 (C-2,7), 171.49 (NCO), 181.22 (CO), 183.15 (CO).

### (22). 2,7-Bis[3-(ethylamino)propionamido]anthraquinone (Compound 22)

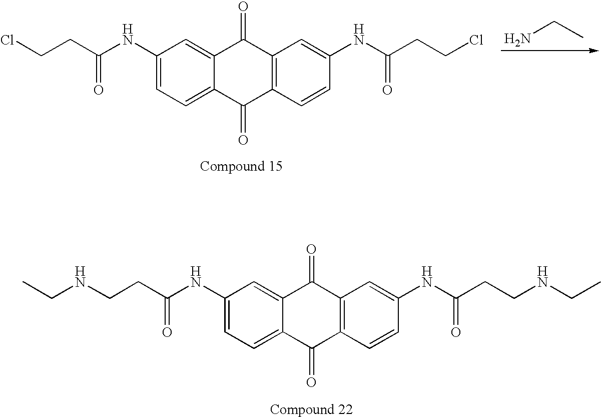

Compound 15 of 1 mmol (0.419 g) was dissolved in 20 ml of N,N-dimethylformamide, and 10 mmol (0.6 ml) of ethylamine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 22 was obtained by re-crystallizing the precipitate with ethanol.

Compound 22 has the following properties: yield: 23%; mp: 181° C. (EtOH); IR (KBr) (cm⁻¹): 1670 (CO), 3332 (NH); ¹H-NMR (300 MHz, CDCl₃) δ (ppm): 2.41 (s, 10H, CH₂CH₃), 2.53 (t, J=5.3 Hz, 4H, CH₂N), 2.67 (t, J=5.3 Hz, 4H, COCH₂), 8.01 (s, 2H, H-1,8), 8.23 (s, 4H, H-3,4,5,6), 11.68 (s, 2H, NH); ¹³C-NMR (300 MHz, DMSO) δ (ppm): 29.67 (CH₃), 33.38 (COCH₂), 44.38 (NCH₂), 54.88 (CH₂N), 116.85 (C-1,8), 124.67 (C-3,6), 129.05 (C-4,4a,5,5a), 134.68 (C-4a,9a), 144.02 (C-2,7), 171.47 (NCO), 180.03 (CO), 182.07 (CO).

### (23). 2,7-Bis[3-(N-methylpiperazino)propionamido]anthraquinone (Compound 23)

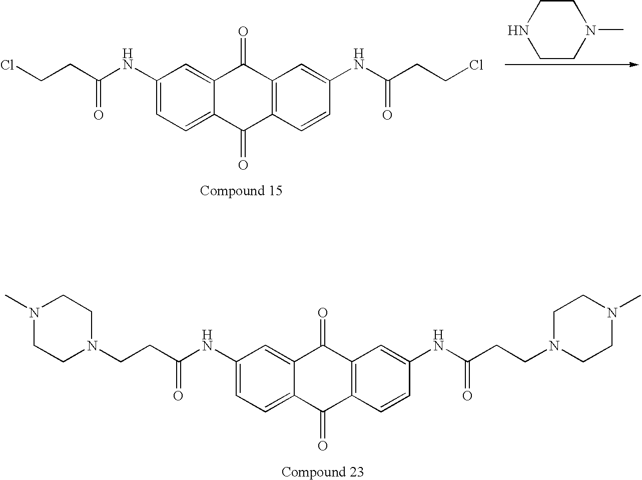

Compound 15 of 1 mmol (0.419 g) was dissolved in 20 ml of N,N-dimethylformamide, and 10 mmol (1.1 ml) of N-methylpiperazine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 23 was obtained by re-crystallizing the precipitate with ethanol.

Compound 23 has the following properties: yield: 61%; mp: 284° C. (EtOH); IR (KBr) (cm⁻¹): 1669 (CO), 1701 (CONH), 3273 (NH); MS (EI, 70 ev)=546 (M⁺); ¹H-NMR (300 MHz, DMSO) δ (ppm): 2.40 (s, 6H, CH₃), 2.57-2.76 (m, 24H, CH₂CH₂, H-2′,3′,5′,6′), 8.15 (d, J=9.6 Hz, 4H, H-1,3,6,8), 8.26 (d, J=8.1 Hz, 2H, H-4,5), 11.69 (s, 2H, NH); ¹³C-NMR (300 MHz, DMSO) δ (ppm): 23.51 (COCH₂), 32.46 (CH₃), 45.35 (CH₂N), 52.26 (C-2′,6′), 55.32 (C-3′,5′), 116.53 (C-1,8), 124.38 (C-3,6), 129.13 (C-4a,5a), 129.21 (C-4,5), 134.55 (C-4a,9a), 144.10 (C-2,7), 171.12 (NCO), 181.29 (CO), 182.51 (CO).

### (24). 2,7-Bis[3-(piperazino)propionamido]anthraquinone (Compound 24)

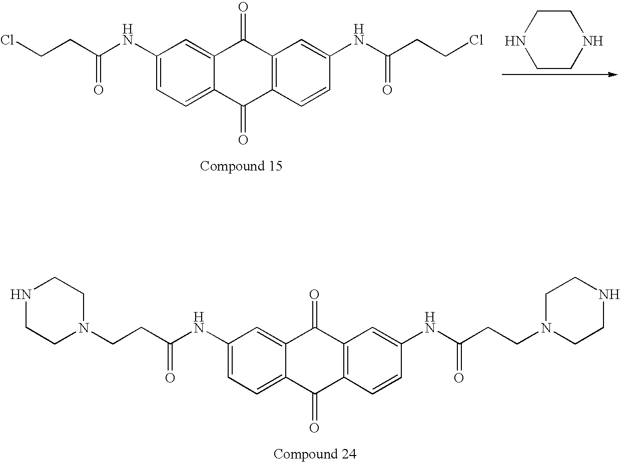

Compound 15 of 1 mmol (0.419 g) was dissolved in 20 ml of N,N-dimethylformamide, and 10 mmol (0.9 ml) of piperazine was added. The mixture then was catalyzed with 0.5 ml of pyridine. This mixture was reacted in a closed device (mini-reactor) under the oil bath at 130 to 150° C. for 30 to 50 minutes. The reacted mixture was added into a small amount of crushed ice, and the precipitate appeared now. The precipitate was obtained by filtration. Finally, Compound 24 was obtained by re-crystallizing the precipitate with ethanol.

Compound 24 has the following properties: yield: 41%; mp: 218° C. (EtOH); IR (KBr) (cm⁻¹): 1671 (CO), 1694 (CONH), 3338 (NH); ¹H-NMR (300 MHz, DMSO) δ (ppm): 2.33 (s, 8H, H-3′,5′), 2.54 (d, J=5.7 Hz, 4H, CH₂N), 2.60 (d, J=5.7 Hz, 4H, COCH₂), 2.67 (t, J=4.5 Hz, 8H, H-2′,6′), 8.02 (dd, J=8.4, 2.1 Hz, 2H, H-3,6), 8.13 (d, J=8.4 Hz, 2H, H-4,5), 8.42 (d, J=2.1 Hz, 2H, H-1,8), 10.75 (s, 2H, NH); ¹³C-NMR (300 MHz, DMSO) δ (ppm): 33.60 (COCH₂), 45.13 (C-3′,5′), 53.375 (CH₂N), 53.77 (C-2′,6′), 115.36 (C-1,8), 123.32 (C-3,6), 127.61 (C-4a,5a), 128.07 (C-4,5), 133.91 (C-8a,9a), 144.05 (C-2,7), 171.03 (NCO), 180.08 (CO), 182.23 (CO).

### (25). 2,7-Bis(4-chlorobutyramido)anthraquinone (Compound 25)

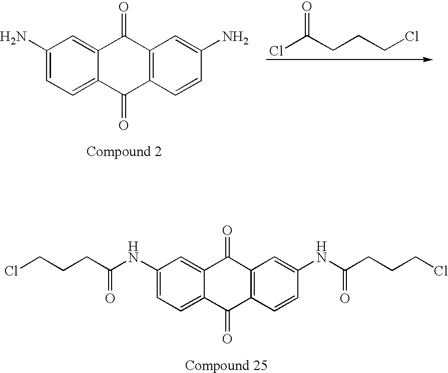

Compound 2 of 1 mmol (0.238 g) was dissolved in 20 ml of N,N-dimethylformamide, and 0.5 ml of pyridine was added to catalyze Compound 2 under the ice bath. Then, 4-chlorobutyryl chloride of 6 mmol (0.7 ml) was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 25 was obtained by re-crystallizing the precipitate with ethanol.

Compound 25 has the following properties: yield: 73%; mp: 234° C. (EtOH); IR (KBr) (cm⁻¹): 1645, 1664 (CO), 1698 (CONH), 3345 (NH); MS (EI, 70 ev)=446.0 (M⁺); ¹H-NMR (300 MHz, DMSO) δ (ppm): 2.05 (m, 4H, CH₂), 2.54 (t, J=7.2 Hz, 4H, COCH₂), 3.71 (t, J=6.5 Hz, 4H, CH₂Cl), 7.97 (dd, J=8.7, 2.1 Hz, 2H, H-3,6), 8.05 (d, J=8.7 Hz, 2H, H-4,5), 8.36 (d, J=1.8 Hz, 2H, H-1,8), 10.54 (s, 2H, NH); ¹³C-NMR (300 MHz, DMSO) δ (ppm): 21.18 (CH₂), 26.86 (COCH₂), 67.72 (CH₂Cl), 110.74 (C-1,8), 119.04 (C-3,6), 122.88 (C-4a,5a), 128.34 (C-4,5), 134.24 (C-4a,9a), 150.95 (C-2,7), 177.22 (NCO), 178.72 (CO), 183.24 (CO).

### (26). 2,7-Bis(acetamido)anthraquinone (Compound 26)

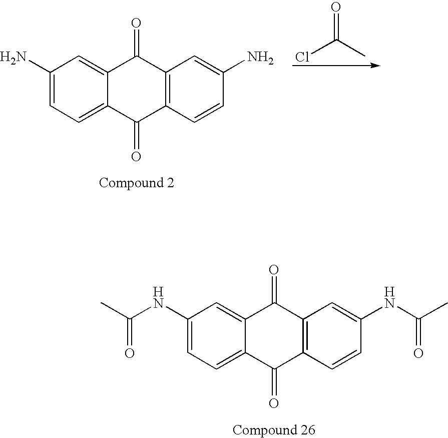

Compound 2 of 1 mmol (0.238 g) was dissolved in 20 ml of N,N-dimethylformamide, and 0.5 ml of pyridine was added to catalyze Compound 2 under the ice bath. Acetyl chloride of 6 mmol (0.5 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 26 was obtained by re-crystallizing the precipitate with ethanol.

Compound 26 has the following properties: yield: 42%; mp: 340° C. (EtOH); IR (KBr) (cm⁻¹): 1645, 1670 (CO), 1691 (CONH), 3333 (NH); MS (EI, 70 ev)=322.1 (M⁺); ¹H-NMR (300 MHz, DMSO) δ (ppm): 2.21 (s, 6H, CH₃), 8.00 (dd, J=8.7, 2.1 Hz, 2H, H-3,6), 8.09 (d, J=8.7 Hz, 2H, H-4,5), 8.38 (d, J=2.1 Hz, 2H, H-1,8), 10.54 (s, 2H, NH); ¹³C-NMR (300 MHz, DMSO) δ (ppm): 23.59 (CH₃), 115.02 (C-1,8), 122.81 (C-3,6), 127.21 (C-4a,5a), 127.51 (C-4,5), 133.42 (C-4a,9a), 143.76 (C-2,7), 168.56 (NCO), 179.52 (CO), 181.67 (CO).

### (27). 2,7-Bis(propionamido)anthraquinone (Compound 27)

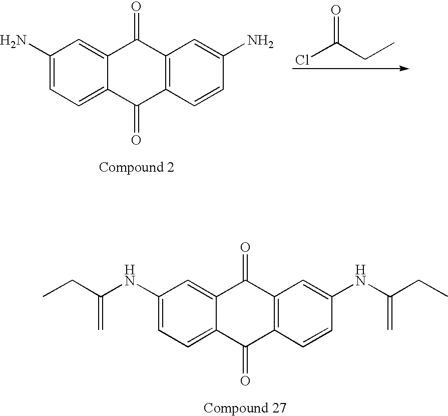

Compound 2 of 1 mmol (0.238 g) was dissolved in 20 ml of N,N-dimethylformamide, and 0.5 ml of pyridine was added to catalyze Compound 2 under the ice bath. Then, propionyl chloride of 6 mmol (0.5 ml) was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 27 was obtained by re-crystallizing the precipitate with ethanol.

Compound 27 has the following properties: yield: 48%; mp: 291° C. (EtOH); IR (KBr) (cm⁻¹): 1672 (CO), 1706 (CONH), 3368 (NH); MS (EI, 70 ev)=350.1 (M⁺); ¹H-NMR (300 MHz, DMSO) δ (ppm): 1.10 (t, J=7.5 Hz, 6H, CH₃), 2.39 (q, J=7.5 Hz, 4H, CH₂), 8.02 (dd, J=8.7, 2.1 Hz, 2H, H-3,6), 8.10 (d, J=8.7 Hz, 2H, H-4,5), 8.41 (d, J=1.8 Hz, 2H, H-1,8), 10.46 (s, 2H, NH); ¹³C-NMR (300 MHz, DMSO) δ (ppm): 8.72 (CH₃), 29.12 (CH₂), 115.12 (C-1,8), 122.93 (C-3,6), 127.20 (C-4a,5a), 127.55 (C-4,5), 133.50 (C-4a,9a), 143.87 (C-2,7), 172.26 (NCO), 179.60 (CO), 181.77 (CO).

### (28). 2,7-Bis(butyramido)anthraquinone (Compound 28)

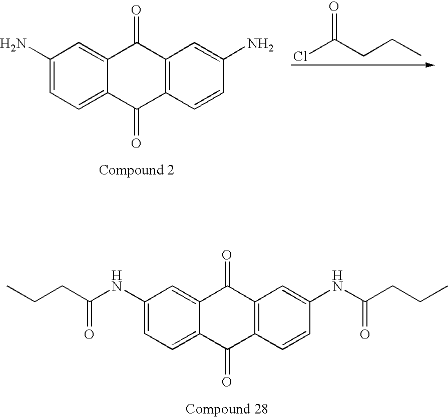

Compound 2 of 1 mmol (0.238 g) was dissolved in 20 ml of N,N-dimethylformamide, and 0.5 ml of pyridine was added to catalyze Compound 2 under the ice bath. Butyryl chloride of 6 mmol (0.6 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 28 was obtained by re-crystallizing the precipitate with ethanol.

Compound 28 has the following properties: yield: 63%; mp: 275° C. (EtOH); IR (KBr) (cm⁻¹): 1664, 1674 (CO), 1706 (CONH), 3339 (NH); MS (EI, 70 ev)=378.2 (M⁺); ¹H-NMR (300 MHz, DMSO) δ (ppm): 0.93 (t, J=7.5 Hz, 6H, CH₃), 1.63 (m, 4H, CH₂), 2.36 (t, J=7.2 Hz, 4H, COCH₂), 8.03 (dd, J=8.4, 2.1 Hz, 2H, H-3,6), 8.10 (d, J=8.4 Hz, 2H, H-4,5), 8.43 (d, J=2.1 Hz, 2H, H-1,8), 10.48 (s, 2H, NH); ¹³C-NMR (300 MHz, DMSO) δ (ppm): 12.97 (CH₃), 17.73 (CH₂), 37.86 (COCH₂), 115.11 (C-1,8), 122.87 (C-3,6), 127.19 (C-4a,5a), 127.48 (C-4,5), 133.44 (C-4a,9a), 143.77 (C-2,7), 171.36 (NCO), 179.50 (CO), 181.69 (CO).

### (29). 2,7-Bis(benzoamido)anthraquinone (Compound 29)

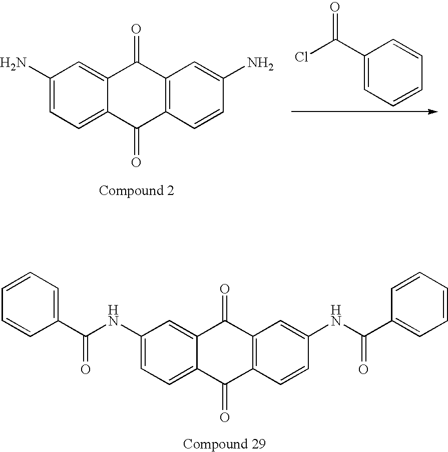

Compound 2 of 1 mmol (0.238 g) was dissolved in 20 ml of N,N-dimethylformamide, and 0.5 ml of pyridine was added to catalyze Compound 2 under the ice bath. Benzoyl chloride of 6 mmol (0.7 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 29 was obtained by re-crystallizing the precipitate with ethanol.

Compound 29 has the following properties: yield: 30%; mp: 224° C. (EtOH); IR (KBr) (cm⁻¹): 1678 (CO), 1703 (CONH), 3320 (NH); MS (EI, 70 ev)=446.1 (M⁺); ¹H-NMR (300 MHz, DMSO) δ (ppm): 7.57 (m, 6H, H-3′,4′,5′), 8.00 (d, J=8.1 Hz, 4H, H-2′,6′), 8.15 (d, J=8.4 Hz, 2H, H-4,5), 8.30 (dd, J=8.4, 1.8 Hz, 2H, H-3,6), 8.65 (d, J=2.1 Hz, 2H, H-1,8), 10.80 (s, 2H, NH); ¹³C-NMR (300 MHz, DMSO) δ (ppm): 116.69 (C-1,8), 124.39 (C-3,6), (C-2′,6′), 127.68 (C-3′,4′,5′), 127.81 (C-4a,5a), 128.03 (C-4,5), 131.66 (C-4a,9a), 133.73 (C-1′), 144.15 (C-2,7), 165.74 (NCO), 180.12 (CO), 182.14 (CO).

### (30). 2,7-Bis(phenylacetamido)anthraquinone (Compound 30)

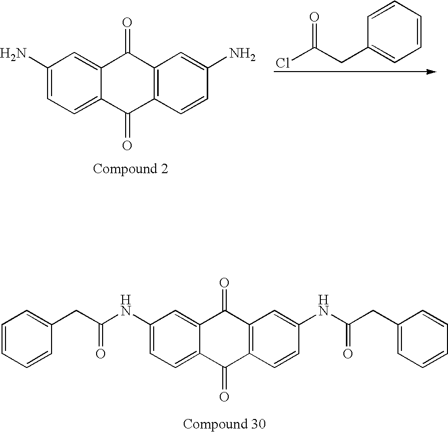

Compound 2 of 1 mmol (0.238 g) was dissolved in 20 ml of N,N-dimethylformamide, and 0.5 ml of pyridine was added to catalyze Compound 2 under the ice bath. Phenylacetyl chloride of 6 mmol (0.8 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 30 was obtained by re-crystallizing the precipitate with ethanol.

Compound 30 has the following properties: yield: 74%; mp: 224° C. (EtOH); IR (KBr) (cm⁻¹): 1644, 1668 (CO), 3322 (NH); MS (EI, 70 ev)=474.2 (M⁺); ¹H-NMR (300 MHz, DMSO) δ (ppm): 3.72 (s, 4H, COCH₂), 7.30 (m, 10H, H-2′,3′,4′,5′,6′), 8.05 (dd, J=8.4, 2.1 Hz, 2H, H-3,6), 8.13 (d, J=8.4 Hz, 2H, H-4,5), 8.44 (d, J=2.1 Hz, 2H, H-1,8), 10.79 (s, 2H, NH); ¹³C-NMR (300 MHz, DMSO) δ (ppm): 42.83 (CH₂), 115.54 (C-1,8), 123.42 (C-3,6), 126.26 (C-3′,4′,5′), 127.73 (C-4a,5a), 128.74 (C-2′,6′), 133.80 (C-4,5), 134.95 (C-1′), 143.97 (C-2,7), 169.72 (NCO), 180.01 (CO), 182.08 (CO).

### (31). 2,7-Bis(phenylpropionamido)anthraquinone (Compound 31)

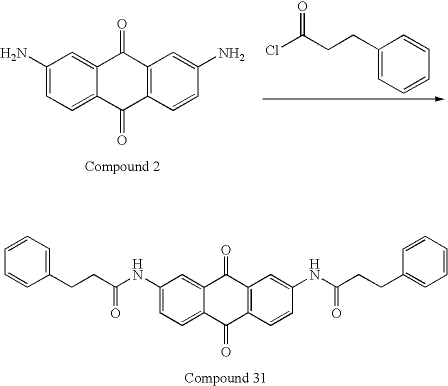

Compound 2 of 1 mmol (0.238 g) was dissolved in 20 ml of N,N-dimethylformamide, and 0.5 ml of pyridine was added to catalyze Compound 2 under the ice bath. Phenylpropionyl chloride of 6 mmol (0.9 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 31 was obtained by re-crystallizing the precipitate with ethanol.

Compound 31 has the following properties: yield: 45; mp: 268° C. (EtOH); IR (KBr) (cm⁻¹): 1672 (CO), 3336 (NH); MS (EI, 70 ev)=502.1 (M⁺); ¹H-NMR (300 MHz, DMSO) δ (ppm): 2.71 (t, J=7.8 Hz, 4H, CH₂), 2.94 (t, J=7.8 Hz, 4H, COCH₂), 7.23 (m, 10H, H-2′,3′,4′,5′,6′), 8.02 (dd, J=8.4, 2.1 Hz, 2H, H-3,6), 8.12 (d, J=8.4 Hz, 2H, H-4,5), 8.43 (d, J=2.1 Hz, 2H, H-1,8), 10.53 (s, 2H, NH); ¹³C-NMR (300 MHz, DMSO) δ (ppm): 30.10 (CH₂), 41.17 (COCH₂), 115.47 (C-1,8), 123.41 (C-3,6), 125.73 (C-2′,6′), 127.68 (C-3′,4′,5′), 127.96 (C-4a,5a), 128.07 (C-4,5), 133.94 (C-4a,9a), 140.65 (C-1′), 144.09 (C-2,7), 171.22 (NCO), 180.15 (CO), 182.30 (CO).

### (32). 2,7-Bis(cyclopropanecarbonamido)anthraquinone (Compound 32)

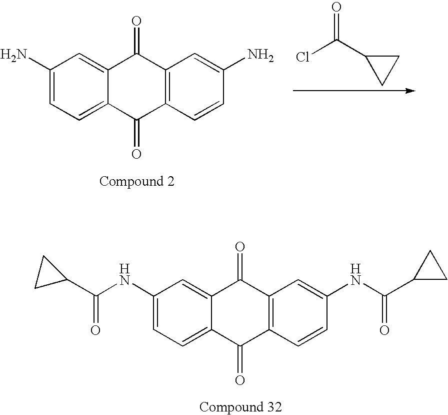

Compound 2 of 1 mmol (0.238 g) was dissolved in 20 ml of N,N-dimethylformamide, and 0.5 ml of pyridine was added to catalyze Compound 2 under the ice bath. Cyclopropanecarbonyl chloride of 6 mmol (0.6 ml) then was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 32 was obtained by re-crystallizing the precipitate with ethanol.

Compound 32 has the following properties: yield: 68%; mp: 352° C. (EtOH); IR (KBr) (cm⁻¹): 1663 (CO), 3292 (NH); MS (EI, 70 ev)=374.9 (M⁺); ¹H-NMR (300 MHz, DMSO) δ (ppm): 0.86 (d, J=6.3 Hz, 8H, H-2′,3′), 1.83 (m, 2H, COCH), 8.02 (dd, J=8.7, 2.1 Hz, 2H, H-3,6), 8.10 (d, J=8.7 Hz, 2H, H-4,5), 8.42 (d, J=2.1 Hz, 2H, H-1,8), 10.80 (s, 2H, NH); ¹³C-NMR (300 MHz, DMSO) δ (ppm): 7.45 (C-2′,3′), 14.37 (C-1′), 115.38 (C-1,8), 123.25 (C-3,6), 127.53 (C-4a,5a), 128.05 (C-4,5), 133.92 (C-4a,9a), 144.11 (C-2,7), 172.45 (NCO), 180.09 (CO), 182.27 (CO).

### (33). 2,7-Bis(cyclopentanecarbonamido)anthraquinone (Compound 33)

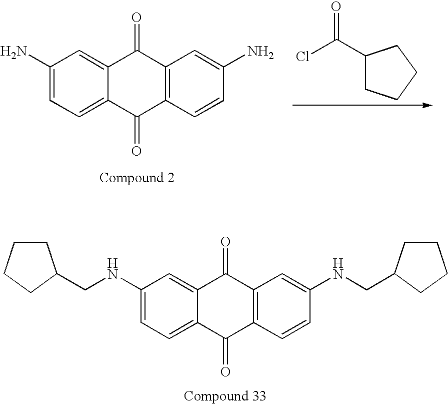

Compound 2 of 1 mmol (0.238 g) was dissolved in 20 ml of N,N-dimethylformamide, and 0.5 ml of pyridine was added to catalyze Compound 2 under the ice bath. Cyclopentanecarbonyl chloride of 6 mmol (0.7 ml) was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 33 was obtained by re-crystallizing the precipitate with ethanol.

Compound 33 has the following properties: yield: 51%; mp: 281° C. (EtOH); IR (KBr) (cm⁻¹): 1647, 1674 (CO), 1702 (CONH), 3342 (NH); MS (EI, 70 ev)=430.9 (M⁺); ¹H-NMR (300 MHz, DMSO) δ (ppm): 1.54-1.89 (m, 16H, H-2′,3′,4′,5′), 2.83 (m, 2H, COCH), 8.04 (dd, J=8.4, 2.1 Hz, 2H, H-3,6), 8.22 (d, J=8.4 Hz, 2H, H-4,5), 8.46 (d, J=2.1 Hz, 2H, H-1,8), 10.47 (s, 2H, NH); ¹³C-NMR (300 MHz, DMSO) δ (ppm): 25.14 (C-3′,4′), 29.45 (C-2′,5′), 44.95 (C-1′), 115.42 (C-1,8), 123.20 (C-3,6), 127.39 (C-4a,5a), 127.80 (C-4,5), 133.70 (C-4a,9a), 144.22 (C-2,7), 174.95 (NCO), 179.92 (CO), 182.12 (CO).

### (34). 2,7-Bis(cyclohexanecarbonamido)anthraquinone (Compound 34)

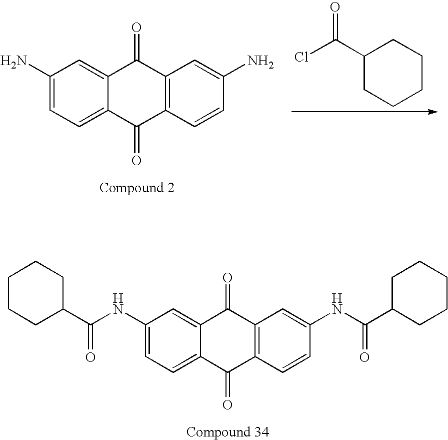

Compound 2 of 1 mmol (0.238 g) was dissolved in 20 ml of N,N-dimethylformamide, and 0.5 ml of pyridine was added to catalyze Compound 2 under the ice bath. Cyclohexanecarbonyl chloride of 6 mmol (0.8 ml) was added, the ice bath was removed, nitrogen gas was injected, and the light-avoided stirring reaction was performed at room temperature for 1 day. The reacted mixture was added into a small amount of crushed ice, and the precipitate appeared now. The precipitate was obtained by filtration, and washed with diethyl ether. Finally, Compound 34 was obtained by re-crystallizing the precipitate with ethanol.

Compound 34 has the following properties: yield: 42%; mp: 269° C. (EtOH); IR (KBr) (cm⁻¹): 1670 (CO), 3323 (NH); MS (EI, 70 ev)=458.9 (M⁺); ¹H-NMR (300 MHz, DMSO) δ (ppm): 1.16-1.46 (m, 12H, H-3′,4′,5′), 1.63-1.84 (m, 8H, H-2′,6′), 2.37 (m, 2H, COCH), 8.02 (dd, J=8.4, 1.8 Hz, 2H, H-3,6), 8.09 (d, J=8.4 Hz, 2H, H-4,5), 8.44 (d, J=1.8 Hz, 2H, H-1,8), 10.43 (s, 2H, NH); ¹³C-NMR (300 MHz, DMSO) δ (ppm): 24.53 (C-3′,5′), 24.76 (C-4′), 28.37 (C-2′,6′), 44.42 (C-1′), 115.47 (C-1,8), 123.29 (C-3,6), 127.43 (C-4a,5a), 127.76 (C-4,5), 133.73 (C-4a,9a), 144.26 (C-2,7), 174.82 (NCO), 179.97 (CO), 182.15 (CO).

In order to determine the telomerase-inhibition capacity of the compounds provided by the present invention to the cancer cells, the pharmacological activity is further analyzed in accordance with the above-mentioned 34 anthraquinone compounds. The analytic method includes the secreted alkaline phosphatase (SEAP) assay and the telomeric repeat amplification protocol (TRAP). The steps and the results of the analytic methods are described respectively as follows.

### I. Secreted Alkaline Phosphatase (SEAP) Assay

The condition of the cells for selecting the telomerase inhibitor must be the cancer cells with telomerase activity. H1299 is a non-small cell lung cancer cell line with telomerase activity. The hTERT communication gene linked behind P_{hTERT} which is located at the fifth chromosome of H1299 cell can be expressed; however, this expression is hard to detect. Therefore, the plasmid having the SEAP communication gene linked behind P_{hTERT} was transfected into H1299 cells. Under the identical condition, after P_{hTERT} was activated, the SEAP communication gene linked behind the plasmid was also activated. This gene could be detected by this analytic method while expressing.

If additionally added reagent can inhibit P_{hTERT}, the linked SEAP communication gene will not be expressed, and the hTERT communication gene linked behind P_{hTERT} at the fifth chromosome will not be expressed.

1. Preparation of Reagents:

(1). Phosphate-buffered saline (PBS) was prepared by dissolving 0.8 g of NaCl, 0.2 g of KCl, 0.61 g of NaHPO₄ (anhydrous) and 0.2 g of KH₂PO₄ to a final volume of 1 liter and adjusting pH to 7.0-7.4.

(2). Trypsin solution was prepared by dissolving trypsin powder in 100 ml of PBS containing 0.25% of EDTA and 0.05% of glucose. The trypsin solution then was sterilized by 0.02 nm-pore size filter and stored at 0° C.

(3). Lysis buffer was prepared by dissolving 50 g of sodium dodecyl sulfate (SDS) in 250 ml of 50% dimethylformamide (DMF), and was stored at 4° C. and light-avoided.

(4). Secreted alkaline phosphatase (SEAP) buffer was obtained by mixing 2 M of diethanolamine with 1 mM of MgCl₂ and 20 mM of L-homoarginine.

(5). 3-(4,5-di-methylthiazol)-2,5-di-phenyltetrazolium bromide (MTT) solution was prepared by dissolving 0.5 g of MTT powder in 50 ml of PBS solution to achieve 5 mg/ml, and was stored at 4° C. and light-avoided.

(6). p-Nitrophenylphosphate (PnPP) solution was prepared by dissolving 31.6 mg of p-nitrophenylphosphate in 1 ml of distilled water.

2. Cell Lines:

H1299 is a non-small cell lung cancer cell line with telomerase activity. The cell extract of H1299 cell lines can be used for selecting the telomerase activity inhibitors in vitro and the telomerase activity-inhibited substances in vivo. H1299 cell line was incubated with RPMI 1640 medium supplemented with 10% of fetal bovine serum (FBS).

3. Cell Count and Surviving Test:

Fifty (50) μl of 0.4% trypan blue was mixed well with 150 μl of fresh medium. A few mixture was injected from the upper groove of hemocytometer, and observed under the reverse microscope with a magnification of 100 times. The living cells were not stained, but the dead ones were stained as blue. The cell numbers were counted within 8 large squares, subsequently divided by 8, multiplied with the dilution times of 5, and finally multiplied with 10⁴. Therefore, the cell number per milliliter in the cellular suspension was calculated. If the cells were located on the lines of the hemocytometer, only cells on the upper and right lines were counted.

4. Selecting Analysis of Telomerase Inhibitor Using SEAP System:

A total of 2×10³ hTERT-BJ1-P_{TERT}(3.4)-SEAP cells were seeded in the 96-well cell culture plate, and incubated with DMEM (GIBCO®)/glucose mixture and Medium 199 (GIBCO®)/10% FBS/1 mM sodium pyruvate (GIBCO®)/4 mM L-glutamine (GIBCO®) mixture at a ratio of 4:1 at 37° C. for 24 hours. When the cells were attached in the bottom of the well, the seriously-diluted compounds with different concentrations (the final concentration were 0.01, 0.1 and 1 mg/ml respectively) were treated with the cells for 48 hours, and the P_{TERT}(3.4)-SEAP cells without drug treatment were being the control. After 48 hours of drug treatment, the cultured medium were collected for performing the analysis of SEAP activity. The cells were washed with 1×PBS once immediately, and the MTT assay was performed to compare the compounds with the relative toxicity or the influence to the proliferation and activity.

5. Analysis of SEAP Activity:

The cellular medium after the cellular incubation was transferred into the eppondorf tube, and the activity of endogeneous alkaline phosphatase was inactivated at 65° C. for 10 minutes. After centrification, the mixture was obtained by mixing 50 μl of the supernatant with the equal volume of the SEAP buffer. The mixture and the substrate (120 mM of p-nitrophenylphosphate) were preheated at 37° C. for 10 minutes respectively, and the mixture and the substrate were mixed at a ratio of 1:10 and reacted at 37° C. The absorbance of visible light at 405 nm was determined at the appropriate time intervals, and the enzymatic activity of SEAP was represented by the speed of the increasing absorbance at 405 nm.

6. MTT Assay:

Mitochondria is the place for cellular respiration, and there are many reductive reactions catalyzed by dehydrogenases. MTT is a water-soluble yellow compound, and the crystal violet will be formed after reduction. This crystal can be dissolved in the organic solvent, and has high absorbance at the wavelength of 570 nm. The higher cellular activity or more cell numbers, the more generated crystal violets. Therefore, the cellular activity and cell number can be obtained by the MTT assay.

MTT solution was prepared by dissolving MTT powder in PBS solution to achieve 5 mg/ml, filtrated and stored at 4° C. Lysis buffer contained 20% (w/v) of SDS in 50% of N,N-dimethylformamide (DMF, Riedel-deHaën). After the cells were incubated in the 96-well culture plate, the original medium was discarded, 100 μl of the fresh serum-free medium per well and 25 μl of MTT solution per well were added in respectively, and the cells were further incubated in the CO₂ incubator at 37° C. for 4 hours. Then, 100 μl of lysis buffer per well was added in and incubated overnight in the CO₂ incubator at 37° C. The absorbance at 570 nm was determined by the ELISA reader.

### II. Telomeric Repeat Amplification Protocol (TRAP)

TRAP is the current frequently-used method for detecting telomerase activity. This method includes two major stages. The first stage was the telomerase elongation supplied with the oligonucleic telomeric sequence using TSG4 primer (5′-GGG ATT GGG ATT GGG ATT GGG TT-3′), and the second stage was the polymerase chain reaction (PCR) for abundantly replicating the telomerase product with CX primer (5′-CCC TTA CCC TTA CCC TTA CCC TAA-3′). While the compound has the telomerase inhibition activity, the replication of telomeric sequence will be ceased. A 36-mer oligonucleotide (TSNT: 5′-AAT CCG TCG AGC AGA GTT AAA AGG CCG AGA AGC GAT-3′) was added in the TRAP analytic reaction to be the internal control. This oligonucleotide could use the identical TS primer in the amplification of PCR together with the TRAP reaction, but another reverse primer (NT primer: 5′-ATC GCT TCT CGG CCT TTT-3′) should be added to amplify in the PCR. This control was mainly in detecting the activity of Taq polymerase.

1. TRAP Activity Analysis:

While performing the analytic method, first, 360 nM of CX primer, 185 nM of NT primer and 400 nM of oligonucleotide TSNT were added in the bottom of the eppondorf tube, and AmpliWax PCR Gem 50 (Perkin Elmer) was added therein. The eppondorf tube was heated at 95° C. for 3 minutes and then 70° C. for 30 seconds by the PCR heat cycler machine, and the eppondorf tube with the sealed wax was taken out until the temperature declined to 4° C.

Four (4) μl of the cellular extract to be analyzed contained about 0.5 to 2 μl of the cellular extract total protein (equivalent to the extract with 10³ to 10⁴ cells), and the cellular extract was mixed with 50 μl of the reaction mixtures, which includes 50 uM of dNTP, more than 3000 cpm of the labeled TS primer, 360 nM of the unlabeled TS primer, 1 μg of Taq polymerase and T-PCR buffer (10×T-PCR buffer contains 200 mM of Tris, 15 mM of MgCl₂, 680 mM of KCl, 0.5% of Tween 20 and 10 mM EDTA, pH 8.3). Distilled water used in the reaction should be treated with 0.1% of DEPC for 24 hour, and sterilized by autoclave. RNase in the water could be inactivated for preventing to influence the reaction in this step.

The cellular extract and the reaction reagent were added in a 0.2-ml PCR tube. The reaction was performed at 30° C. for 30 minutes, so as to elongate the TSG4 primer by the telomerase of the analytic cellular extract. Subsequently, the whole reaction mixture was heated at 94° C. for 90 seconds, then the PCR was performed at the conditions of 94° C. for 30 seconds, 50° C. for 30 seconds, 72° C. for 45 seconds, and a total of 35 cycles. Finally, one cycle was performed at 94° C. for 30 seconds, 50° C. for 30 seconds and 72° C. for 1 minute, and the whole reaction was terminated. Five (5) μl of 5 mg/ml RNase A was added in the whole reaction to be the negative control of the TRAP analytic method.

Forty-five (45) μl in 50 μl of the reacted PCR mixture was mixed well with 9 μl of the gel-loading buffer (6× gel-loading buffer contains 0.25% of bromophenol blue, 0.25% of xylene cyanol and 30% of glycerol). Then, 15 μl of the mixture was loaded into 8% TBE polyacrylamide gel (acrylamide:bis-acrylamide=19:1), and electrophoresis was performed at 140 volts for 2 hours. The electrophoresis gel was dried in the gel dryer, and the isotopic automatic irradiation development was proceeded on the X-ray film after the gel was taken off. The telomerase activity was identified from the development result.

### III. Results of Pharmacological Activity Analysis

1. Selection Result of Telomerase Inhibitor in SEAP System

a. Selection Result of Telomerase Inhibitor by SEAP Assay

The selection of telomerase inhibitors of the anthraquinone compounds provided in the present invention was performed by the H1299/P_{hTERT}-driven SEAP system. SEAP is the communication gene in this drug selection model. The SEAP inhibition was shown as follows. The quantitative cellular medium obtained after the 24-hour drug-treatment and the identical inactivation was reacted with the quantitative SEAP substrate, and the absorbance at 405 nm was detected for 5 time intervals. Subsequently, the linear slope of absorbance to the measuring time was calculated, and the variance of absorbance at 405 nm per minute was represented and compared with the SEAP-producing result of drug-untreated H1299 cells. In addition, the influence of the cell-treated drug solution to the cellular proliferation and the drug toxicity to the cells were considered, and the cellular growth was evaluated by the MTT assay. The drug-treated cells were analyzed by the SEAP assay and the data analysis of MTT assay at the same time. Both results were compared so as to delete the influence of cell number or the activity to SEAP, and the dose-dependent phenomenon could be anticipated in the range of drug concentration designed by the laboratory.

In the preliminary selection, the highest concentration was 1000 μM, and 10-fold serial dilutions were done. Therefore, the final concentrations for treating cells were 1 μM, 10 μM and 100 μM, and the drug concentrations were repeated for 5 times. Since the chemically-synthetic drugs have colors thereon, the SEAP value of drug-treatment without cells was also determined to be the background value which was deducted while treating with the drugs. The candidate drug for inhibiting telomerase on the hTERT gene expression level was evaluated and anticipated.

Please refer to Table 1, which is the hTERT expression and the inhibition effect of telomerase activity of 2,7-disubstituted anthraquinone derivatives of the present invention. Among this, the data were the average of at least triplet and the standard deviation of each average was within 20%. In the SEAP data, the average less than 80% represents that the telomerase inhibition activity existed. The experimental result was found that Compounds 30 and 31, which side chains are aromatic-linked, have the inhibition effect (i.e. SEAP <80%), the cellular survival rate is more than 80% in the MTT assay, and the inhibition effect also increases significantly along with the increased concentration of compounds. In particular, Compound 30 represents low cellular cytotoxicity (MTT assay=107±7%) in low concentration (1 μM), and has excellent telomerase inhibition (SEAP 70±6%).

b. Result of MTT Assay:

The cells used in the experiments were non-small cell lung cancer cell line, H1299. In the aspect of cancer cytotoxicity, the cytotoxicities of Compounds 32, 10 and 7 to H1299 cancer cells are better than that to the traditional anti-cancer drug, mitoxantrone, to H1299 cancer cells. Even, the cytotoxicity of Compound 7 in 1 μM is similar with that of mitoxantrone in 180 μM.

2. Analytic Result of TRAP:

a. Selection Result of Telomerase Inhibitor by TRAP Assay:

In the TRAP analytic method, it is theoretically that TSG4 primer being a telomere sequence can form the specific G-quadruplex structure voluntarily in the normal condition. The compounds are anticipated to stabilize this structure and inactivate the function between the telomerase and the telomere, so as to inhibit the telomerase function. However, this experiment cannot identify whether these compounds have direct inhibition on the telomerase. No matter whether the inhibition effect is achieved by stabilizing the G-quadruplex or by directly inhibiting the telomerase, both are the anticipated and achieved goals. It is to find out the compounds having inhibition effect on the telomerase. In the result of gel electrophoresis, the positive control was analyzed by distilled water substituted for the compounds, and the negative control was analyzed by 5 μl of 0.1 mg/ml RNase A substituted for the compounds. The positive control showed many telomere fragments, but the negative control did not. The concentrations of compounds was 100, 10 and 1 μM respectively. The compounds having telomerase inhibition effect were Compounds 26, 28, 29, 6, 8, 10, 13, 14, 16 and 20, and a total of 10 compounds in the present experiment.

In concluding the above-mentioned experimental results, in the SEAP assay, since hTERT is the main expression regulation factor of telomerase, and the SEAP assay is expressed by the inhibition of the compounds to P_{hTERT}, the relative telomerase activity will be also inhibited when P_{hTERT} is inhibited by the compounds. The analytic result was shown that cells have more than 80% survival rate with respect to Compounds 30 and 31 in the MTT assay, and the inhibition effect of the telomerase activity is also significantly increased along with the increasing concentration of the compounds. In particular, Compound 30 had low cytotoxicity (MTT assay=107±7%) but had excellent telomerase inhibition (SEAP=70±6%) in the low concentration (1 μM). Therefore, Compound 30 is undoubtedly one of the excellent telomerase inhibitor.

In the TRAP assay, since TSG4 primer will form the G-quadruplex structure automatically, the telomerase will not extend the end of chromosome when the compounds stabilizes the G-quadruplex. Therefore, the telomere fragment could not be seen on the X-ray film. The analytic result was shown that 10 compounds, Compounds 26, 28, 29, 6, 8, 10, 13, 14, 16 and 20, have the telomerase inhibition.

3. In Vitro Experimental Result

From the in vitro experiments of the Development Therapeutics Program of the Cancer Research Institute, the United States, it was known that the synthetic heterocyclic anthraquinone derivatives of the present invention have different inhibition levels to the different cancer cell lines under the concentration of 1.0×10⁻⁵ M. Please refer to Table 2, which is the result of in vitro cytotoxicity of 2,7-disubstituted anthraquinone derivatives of the present invention.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 1
The hTERT expression and the inhibition effect of telomerase activity of
2,7-disubstitutedanthraquinone derivatives of the present invention
Tested Cell line (Inhibition μM ± SD)^{a}

| | | H1299 | | | | | |
|---|---|---|---|---|---|---|---|
| | | MTT | | | SEAP | | |
| | Compound/ | Compound concentration (μM) | | | | | |
| No. | Side chain structure (R) | 100 | 10 | 1 | 100 | 10 | 1 |
| 1 | 2,7-dinitroanthraquinone | 69 ± 10 | 95 ± 8 | 97 ± 15 | 62 ± 8 | 85 ± 21 | 87 ± 13 |
| 2 | 2,7-diaminoanthraquinone | 65 ± 10 | 80 ± 16 | 90 ± 17 | 63 ± 12 | 75 ± 8 | 87 ± 10 |
| 3 | CH₂Cl | 1 ± 0 | 5 ± 3 | 58 ± 44 | 36 ± 1 | 40 ± 1 | 72 ± 3 |
| 4 | CH₂N(CH₂CH₃)₂ | 25 ± 3 | 47 ± 7 | 101 ± 8 | 37 ± 10 | 61 ± 11 | 90 ± 3 |
| 5 | CH₂NHCH₂CH₂CH₂CH₃ | 15 ± 11 | 17 ± 9 | 91 ± 16 | 20 ± 13 | 26 ± 17 | 77 ± 18 |
| 6 | 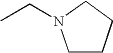 | 23 ± 14 | 52 ± 13 | 99 ± 9 | 30 ± 10 | 48 ± 13 | 88 ± 10 |
| 7 | 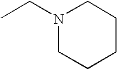 | 35 ± 11 | 37 ± 15 | 50 ± 16 | 63 ± 15 | 69 ± 20 | 74 ± 19 |
| 8 | CH₂N(CH₃)₂ | 15 ± 12 | 55 ± 9 | 92 ± 13 | 21 ± 14 | 40 ± 20 | 87 ± 12 |
| 9 | CH₂NHCH₂CH₂CH₃ | 14 ± 10 | 54 ± 11 | 89 ± 15 | 24 ± 10 | 44 ± 16 | 73 ± 17 |
| 10 | CH₂NHCH₂CH₃ | 3 ± 4 | 24 ± 5 | 52 ± 11 | 34 ± 3 | 35 ± 5 | 60 ± 10 |
| 11 | 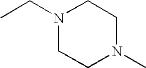 | 37 ± 6 | 71 ± 4 | 90 ± 14 | 23 ± 9 | 50 ± 11 | 76 ± 21 |
| 12 | 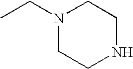 | 88 ± 11 | 85 ± 13 | 88 ± 19 | 60 ± 12 | 72 ± 20 | 89 ± 17 |
| 13 | CH₂NHCH(CH₃)₂ | 6 ± 11 | 26 ± 15 | 78 ± 10 | 21 ± 16 | 28 ± 18 | 73 ± 31 |
| 14 | CH₂NHCH₂CH(CH₃)₂ | 11 ± 5 | 39 ± 10 | 108 ± 8 | 26 ± 10 | 38 ± 15 | 73 ± 20 |
| 15 | CH₂CH₂Cl | 29 ± 11 | 44 ± 15 | 77 ± 13 | 87 ± 9 | 94 ± 7 | 94 ± 11 |
| 16 | CH₂CH₂N(CH₂CH₃)₂ | 1 ± 1 | 41 ± 20 | 106 ± 15 | 26 ± 11 | 35 ± 17 | 76 ± 19 |
| 17 | CH₂CH₂NHCH₂CH₂CH₂CH₃ | 14 ± 10 | 52 ± 4 | 82 ± 12 | 22 ± 17 | 33 ± 9 | 56 ± 14 |
| 18 | 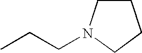 | 12 ± 10 | 54 ± 7 | 87 ± 15 | 17 ± 13 | 29 ± 15 | 73 ± 11 |
| 19 | 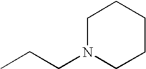 | 18 ± 9 | 44 ± 7 | 87 ± 15 | 25 ± 10 | 33 ± 8 | 55 ± 23 |
| 20 | CH₂CH₂N(CH₃)₂ | 1 ± 1 | 53 ± 13 | 1 ± 104 | 1 ± 22 | 13 ± 30 | 5 ± 94 |
| 21 | CH₂CH₂NHCH₂CH₂CH₃ | 13 ± 10 | 50 ± 6 | 80 ± 15 | 13 ± 15 | 22 ± 14 | 52 ± 13 |
| 22 | CH₂CH₂NHCH₂CH₃ | 34 ± 7 | 59 ± 3 | 86 ± 10 | 22 ± 13 | 33 ± 24 | 68 ± 29 |
| 23 | 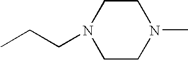 | 39 ± 6 | 80 ± 15 | 95 ± 18 | 21 ± 10 | 52 ± 11 | 75 ± 16 |
| 24 | 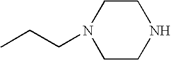 | 102 ± 13 | 94 ± 8 | 97 ± 8 | 71 ± 15 | 71 ± 21 | 75 ± 26 |
| 25 | CH₂CH₂CH₂Cl | 22 ± 2 | 40 ± 2 | 84 ± 18 | 78 ± 17 | 86 ± 13 | 88 ± 19 |
| 26 | CH₃ | 55 ± 11 | 83 ± 13 | 117 ± 15 | 74 ± 18 | 87 ± 14 | 97 ± 10 |
| 27 | CH₂CH₃ | 35 ± 15 | 38 ± 17 | 70 ± 25 | 85 ± 16 | 95 ± 14 | 90 ± 23 |
| 28 | CH₂CH₂CH₃ | 40 ± 15 | 59 ± 13 | 93 ± 14 | 83 ± 4 | 83 ± 6 | 90 ± 8 |
| 29 | 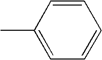 | 41 ± 30 | 47 ± 13 | 71 ± 24 | 85 ± 19 | 92 ± 11 | 104 ± 10 |
| 30 | 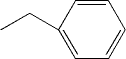 | 75 ± 22 | 96 ± 17 | 106 ± 12 | 80 ± 17 | 91 ± 21 | 97 ± 18 |
| 31 | 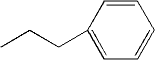 | 76 ± 18 | 94 ± 13 | 102 ± 11 | 61 ± 13 | 79 ± 8 | 89 ± 23 |
| 32 | 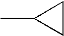 | 43 ± 15 | 36 ± 13 | 46 ± 14 | 81 ± 1 | 81 ± 1 | 80 ± 1 |
| 33 | 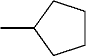 | 42 ± 12 | 77 ± 6 | 91 ± 12 | 85 ± 13 | 87 ± 2 | 83 ± 5 |
| 34 | 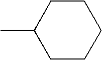 | 52 ± 16 | 63 ± 13 | 90 ± 3 | 80 ± 2 | 93 ± 3 | 96 ± 5 |

| BJ1 | |
|---|---|
| MTT | SEAP |

| | Compound/ | Compound concentration (μM) | | | | | |
|---|---|---|---|---|---|---|---|
| No. | Side chain structure (R) | 100 | 10 | 1 | 100 | 10 | 1 |
| 1 | 2,7-dinitroanthraquinone | 86 ± 3 | 91 ± 2 | 91 ± 2 | 65 ± 23 | 60 ± 35 | 55 ± 21 |
| 2 | 2,7-diaminoanthraquinone | 80 ± 2 | 83 ± 1 | 95 ± 2 | ND^{b} | ND | ND |
| 3 | CH₂Cl | 88 ± 2 | 89 ± 3 | 105 ± 2 | 135 ± 21 | 69 ± 34 | ND |
| 4 | CH₂N(CH₂CH₃)₂ | 50 ± 1 | 67 ± 2 | 116 ± 4 | 116 ± 34 | 131 ± 57 | 26 ± 4 |
| 5 | CH₂NHCH₂CH₂CH₂CH₃ | 43 ± 1 | 41 ± 1 | 103 ± 1 | 80 ± 14 | 24 ± 24 | 27 ± 18 |
| 6 | 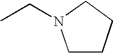 | 39 ± 1 | 60 ± 2 | 99 ± 2 | ND | ND | ND |
| 7 | 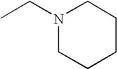 | 22 ± 2 | 78 ± 3 | 89 ± 1 | 103 ± 5 | 121 ± 3 | 118 ± 16 |
| 8 | CH₂N(CH₃)₂ | 39 ± 1 | 45 ± 1 | 96 ± 3 | 3 ± 13 | 44 ± 5 | 96 ± 1 |
| 9 | CH₂NHCH₂CH₂CH₃ | 42 ± 1 | 44 ± 0 | 96 ± 3 | ND | ND | ND |
| 10 | CH₂NHCH₂CH₃ | 37 ± 1 | 56 ± 2 | 72 ± 2 | 17 ± 21 | 41 ± 4 | 90 ± 5 |
| 11 | 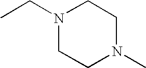 | 47 ± 3 | 69 ± 1 | 86 ± 1 | 97 ± 30 | 68 ± 21 | 61 ± 21 |
| 12 | 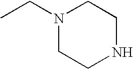 | 76 ± 2 | 85 ± 15 | 88 ± 2 | ND | ND | ND |
| 13 | CH₂NHCH(CH₃)₂ | 39 ± 1 | 41 ± 2 | 89 ± 1 | 45 ± 11 | 55 ± 19 | 79 ± 14 |
| 14 | CH₂NHCH₂CH(CH₃)₂ | 39 ± 0 | 37 ± 0 | 107 ± 3 | 40 ± 3 | 44 ± 2 | 83 ± 5 |
| 15 | CH₂CH₂Cl | 39 ± 1 | 65 ± 1 | 77 ± 2 | ND | ND | ND |
| 16 | CH₂CH₂N(CH₂CH₃)₂ | 36 ± 1 | 55 ± 3 | 103 ± 2 | 37 ± 5 | 49 ± 3 | 84 ± 5 |
| 17 | CH₂CH₂NHCH₂CH₂CH₂CH₃ | 40 ± 0 | 70 ± 1 | 80 ± 3 | 111 ± 25 | 95 ± 46 | 88 ± 34 |
| 18 | 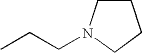 | 39 ± 0 | 68 ± 0 | 82 ± 4 | 109 ± 18 | 67 ± 16 | ND |
| 19 | 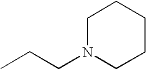 | 41 ± 2 | 54 ± 2 | 92 ± 6 | 234 ± 20 | 226 ± 51 | 186 ± 47 |
| 20 | CH₂CH₂N(CH₃)₂ | 22 ± 13 | 30 ± 14 | 94 ± 7 | 38 ± 0 | 61 ± 2 | 86 ± 2 |
| 21 | CH₂CH₂NHCH₂CH₂CH₃ | 38 ± 2 | 65 ± 2 | 75 ± 1 | 217 ± 40 | 133 ± 48 | ND |
| 22 | CH₂CH₂NHCH₂CH₃ | 42 ± 1 | 73 ± 3 | 82 ± 2 | 97 ± 87 | 217 ± 133 | 211 ± 38 |
| 23 | 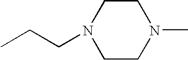 | 61 ± 1 | 80 ± 2 | 95 ± 2 | 232 ± 66 | 138 ± 115 | ND |
| 24 | 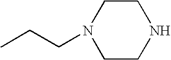 | 88 ± 4 | 100 ± 3 | 96 ± 2 | 131 ± 29 | 118 ± 26 | 163 ± 22 |
| 25 | CH₂CH₂CH₂Cl | 58 ± 3 | 62 ± 2 | 72 ± 3 | 75 ± 16 | 42 ± 45 | 13 ± 58 |
| 26 | CH₃ | 91 ± 2 | 91 ± 2 | 106 ± 1 | 117 ± 5 | 117 ± 3 | 123 ± 5 |
| 27 | CH₂CH₃ | 48 ± 2 | 57 ± 2 | 77 ± 2 | 51 ± 8 | 64 ± 8 | 78 ± 10 |
| 28 | CH₂CH₂CH₃ | 50 ± 6 | 51 ± 8 | 84 ± 2 | ND | ND | ND |
| 29 | 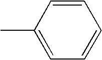 | 97 ± 3 | 85 ± 2 | 92 ± 2 | 36 ± 13 | 72 ± 8 | 89 ± 2 |
| 30 | 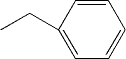 | 86 ± 4 | 80 ± 3 | 93 ± 2 | 40 ± 15 | 79 ± 6 | 85 ± 8 |
| 31 | 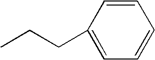 | 44 ± 1 | 44 ± 0 | 89 ± 2 | 43 ± 75 | 92 ± 45 | 74 ± 29 |
| 32 | 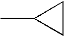 | 47 ± 2 | 44 ± 10 | 60 ± 6 | ND | ND | ND |
| 33 | 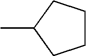 | 58 ± 1 | 68 ± 1 | 77 ± 1 | 108 ± 15 | 130 ± 15 | 137 ± 15 |
| 34 | 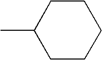 | 64 ± 2 | 76 ± 3 | 87 ± 2 | 22 ± 7 | 14 ± 53 | 14 ± 67 |

^{a}SD: standard deviation;
^{b}ND: not determined.

TABLE 2
The result of in vitro cytotoxicity of 2,7-disubstituted anthraquinone
derivatives of the present invention

| | Cellular growth rate (%) of the Compound | | | | |
|---|---|---|---|---|---|
| Cell line | 8 | 16 | 18 | 26 | 32 |
| Non-small cell | | | | | |
| lung cancer | | | | | |
| EKVX | 102.30 | 59.99 | 58.72 | 95.28 | 94.31 |
| HOP-62 | 28.97 | 11.65 | 18.79 | 110.60 | 91.64 |
| HOP-92 | 56.14 | 9.70 | 7.04 | 101.26 | 86.18 |
| NCl-H226 | 89.14 | 49.89 | 41.10 | 104.51 | 100.60 |
| NCI-H23 | 51.78 | 30.34 | 31.49 | 94.24 | 95.85 |
| NCl-H322M | 32.42 | 44.75 | 45.79 | 97.50 | 105.77 |
| NCl-H460 | 5.01 | 9.50 | 25.52 | 109.29 | 89.03 |
| NCl-H522 | 61.82 | 27.98 | 29.16 | 93.56 | 89.41 |
| Colon cancer | | | | | |
| COLO 205 | −73.57 | −49.69 | −29.04 | 119.85 | 91.92 |
| HCC-2998 | −73.97 | −87.42 | 19.99 | 105.35 | 132.37 |
| HCT-116 | 19.07 | −32.45 | 10.51 | 101.91 | 87.14 |
| HCT-15 | 16.26 | 17.42 | 56.14 | 93.93 | 86.35 |
| HT29 | 11.73 | 3.30 | 19.47 | 107.23 | 106.26 |
| KM12 | 66.95 | 21.82 | 32.55 | 105.94 | 92.67 |
| SW-620 | 23.36 | 16.93 | 20.01 | 103.21 | 104.75 |
| Breast cancer | | | | | |
| BT-549 | 85.36 | 57.04 | 76.91 | 93.29 | 92.51 |
| HS 578T | −6.11 | −23.77 | 5.26 | 133.05 | 118.90 |
| MCF7 | 7.92 | 9.34 | 13.35 | 96.90 | 77.38 |
| MDA-MB- | 4.49 | 31.32 | 30.47 | 103.99 | 97.05 |
| 231/ATCC | | | | | |
| MDA-MB-435 | 68.36 | 71.07 | 68.84 | 109.48 | 97.35 |
| NCI/ADR-RES | 47.84 | 59.52 | 80.59 | 114.26 | 90.83 |
| T-47D | 24.62 | 68.62 | 58.98 | 96.17 | 79.75 |
| Ovarian cancer | | | | | |
| OVCAR-3 | 43.63 | 10.39 | 36.88 | 111.06 | 103.35 |
| OVCAR-4 | 45.70 | 24.92 | 49.82 | 103.60 | 97.54 |
| OVCAR-5 | 73.15 | 33.63 | 18.81 | 102.27 | 112.01 |
| OVCAR-8 | 32.31 | 4.30 | 19.58 | 99.85 | 94.34 |
| SK-OV-3 | 70.26 | 44.02 | 27.14 | 101.12 | 93.16 |
| Leukemia | | | | | |
| CCRF-CEM | −13.21 | −6.02 | 3.74 | 99.77 | 90.67 |
| HL-60(TB) | −64.97 | 14.01 | 85.84 | 38.74 | 62.45 |
| K-562 | 34.91 | 8.10 | 6.40 | 98.54 | 91.60 |
| MOLT-4 | 29.12 | 5.42 | 22.97 | 96.96 | 94.29 |
| RPMI-8226 | 34.97 | −29.02 | 47.90 | 121.47 | 88.38 |
| SR | −46.45 | −0.39 | 19.75 | 3.61 | 1.69 |
| Renal cancer | | | | | |
| 786-0 | 68.60 | 24.72 | 40.51 | 97.72 | 94.92 |
| A498 | −6.39 | −38.76 | −0.91 | 115.60 | 92.35 |
| ACHN | 66.43 | 11.09 | 29.65 | 96.93 | 86.70 |
| CAK1-1 | 59.26 | 3.27 | 55.55 | 105.10 | 88.43 |
| SN12C | 27.99 | 8.63 | 6.68 | 113.35 | 82.93 |
| TK-10 | 73.47 | 17.16 | 31.22 | 118.38 | 136.20 |
| UO-31 | −82.55 | −31.53 | 5.27 | 96.42 | 95.55 |
| Melanoma | | | | | |
| LOX IMVI | −52.34 | 3.39 | 30.74 | 99.05 | 90.27 |
| M14 | 75.04 | 62.79 | 53.88 | 105.11 | 98.89 |
| MALME-3M | 96.21 | 62.39 | 47.00 | 111.26 | 91.76 |
| SK-MEL-2 | −30.35 | −60.38 | −45.21 | 30.15 | 16.85 |
| SK-MEL-28 | 32.11 | 2.56 | 14.99 | 117.60 | 108.51 |
| SK-MEL-5 | 82.59 | 82.75 | 56.94 | 108.87 | 75.67 |
| UACC-257 | −1.84 | −14.46 | −17.88 | 26.97 | 107.63 |
| UACC-62 | 22.31 | 44.45 | 42.72 | 101.24 | 81.00 |
| Prostate cancer | | | | | |
| DU-145 | 78.68 | 10.20 | 29.53 | 116.79 | 98.83 |
| PC-3 | 59.00 | 13.59 | 21.66 | 102.73 | 106.89 |
| CNS cancer | | | | | |
| SF-268 | 64.92 | 35.18 | 26.67 | 113.68 | 95.09 |
| SF-295 | −83.51 | −19.91 | 48.62 | 103.25 | 86.60 |
| SF-539 | −3.72 | 37.15 | 33.75 | 103.04 | 93.24 |
| SNB-19 | 79.57 | 75.84 | 66.97 | 94.40 | 98.79 |
| SNB-75 | 76.12 | 46.62 | 77.47 | 106.91 | 89.23 |
| U251 | 16.26 | 21.65 | 18.16 | 95.14 | 72.62 |
| Mean | 28.70 | 16.33 | 30.97 | 99.06 | 91.54 |
| Delta Value | 112.21 | 103.75 | 76.18 | 95.45 | 89.85 |
| Range | 185.81 | 170.17 | 131.05 | 129.44 | 134.51 |

What is claimed is:

1. A 2,7-disubstituted anthraquinone derivative comprising formula I: 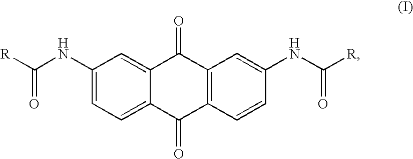
wherein R is a first substituted group selected from a group consisting of a hydrogen, an amino group, a nitro group, a hydroxyl group, a C₁-C₁₂ alkyl group, a C₁-C₁₂ alkyl halide group (—(CH2)ₙX), a C₃-C₁₂ cycloalkyl group, a benzyl group, a C₁-C₁₂ alkylamino group, a C₅-C₁₂ nitrocycloalkyl group and a heterocyclic group, n satisfies 1≦n≦12 and X is an atom selected from a group consisting of a fluoride (F), a chloride (Cl), a bromide (Br) and an iodine (I).

2. The derivative according to claim 1, wherein the C₁-C₁₂ alkyl group is one of a linear C₁-C₁₂ alkyl group and a branched C₁-C₁₂ alkyl group.

3. The derivative according to claim 2, wherein the C₁-C₁₂ alkyl group comprises a methyl group, an ethyl group, a propyl group, a butyl group, an isobutyl group, a pentyl group, an isopentyl group, a heptyl group, an isoheptyl group, an octyl group, an iso-octyl group and a linear alkyl group with a C<5 branched alkyl group.

4. The derivative according to claim 1, wherein the C₁-C₁₂ alkyl halide group comprises a methylhalide group, an ethylhalide group, a propylhalide group, a butylhalide group, an isobutylhalide group, a pentylhalide group, an isopentylhalide group, a heptylhalide group, an isoheptylhalide group, an octylhalide group, an iso-octylhalide group and a linear alkylhalide group with a C<5 branched alkyl group.

5. The derivative according to claim 1, wherein the benzyl group has a para-position, a meta position and an ortho-position, at least one of which is bounded with a second substituted group selected from a group consisting of a hydrogen, a linear C₁-C₃ alkyl group, a branched C₃ alkyl group and a C₁-C₃ alkylamino group.

6. The derivative according to claim 1, wherein the C₃-C₁₂ cycloalkyl group comprises a cyclopropyl group, a cyclopentyl group, a cyclohexyl group, a cycloheptyl group, a cyclo-octyl group, an alkyl ring with a C<5 branched alkyl group, a cyclopropylhalide group, a cyclopentylhalide group, a cyclohexylhalide group, a cycloheptylhalide group, a cyclo-octylhalide group and an alkyl ring with a branched alkylhalide group.

7. The derivative according to claim 6, wherein each one of the C₃-C₁₂ cycloalkyl group has a para-position, a meta position and an ortho-position, at least one of which is bounded with a third substituted group selected from a group consisting of a hydrogen, a branched C₁-C₃ alkyl group and a C₁-C₃ alkylamino group.

8. The derivative according to claim 1, wherein the C₁-C₁₂ alkylamino group is one of a linear C₁-C₁₂ alkylamino group and a branched C₁-C₁₂ alkylamino group.

9. The derivative according to claim 1, wherein each one of the C₅-C₁₂ nitrocycloalkyl group has a para-position, a meta position and an ortho-position, at least one of which is bounded with a fourth substituted group selected from a group consisting of a hydrogen, an amino group, a nitro group, a hydroxyl group, a C₁-C₅ alkyl group, a C<3 branched alkyl group, a C₃-C₅ cycloalkoxyl group, an alkylamino group, a hydroxylhalide group, a C₁-C₅ alkylhalide group, a C<3 branched alkylhalide group, and a C₃-C₅ cycloalkoxylhalide group.

10. The derivative according to claim 1, wherein each one of the heterocyclic group has a para-position, a meta position and an ortho-position, at least one of which is bounded with a fifth substituted group selected from a group consisting of a hydrogen, an amino group, a nitro group, a hydroxyl group, a C₁-C₅ alkyl group, a C<3 branched alkyl group, a C₃-C₅ cycloalkoxyl group, an alkylamino group, a hydroxylhalide group, a C₁-C₅ alkylhalide group, a C<3 branched alkylhalide group, and a C₃-C₅ cycloalkoxylhalide group.

11. A pharmaceutical composition comprising a 2,7-disubstituted anthraquinone derivative as claimed in claim 1.

12. A pharmaceutical composition according to claim 11 being used for treating a cancer and further comprising an additive selected from a group consisting of a pharmaceutically acceptable carrier, a dilutent, an excipient and a combination thereof.

13. The pharmaceutical composition according to claim 12, wherein the 2,7-disubstituted anthraquinone derivative has an effective dose.

14. A pharmaceutical composition according to claim 10 being used for inhibiting a telomerase of a cell and comprising an additive selected from a group consisting of a pharmaceutically acceptable carrier, a dilutent, an excipient and a combination thereof.

15. The pharmaceutical composition according to claim 14, wherein the 2,7-disubstituted anthraquinone derivative has an effective dose.

16. The pharmaceutical composition according to claim 14, wherein the cell is a mammalian cell.

17. A preparation method of a 2,7-disubstituted anthraquinone derivative, comprising steps of:
(a) providing a 2,7-diaminoanthraquinone; and
(b) acetylating the 2,7-diaminoanthraquinone to be bounded with a first side chain having a chloride.

18. The preparation method according to claim 17, wherein the 2,7-diaminoanthraquinone is obtained by steps of:
(a) oxidizing an anthrone to generate a first compound;
(b) nitrifying the first compound to generate a second compound; and
(c) reducing the second compound by a sodium sulfide.

19. The preparation method according to claim 17, wherein the step (b) further comprises steps of:
(b1) dissolving the 2,7-diaminoanthraquinone with an N,N-dimethylformamide;
(b2) catalyzing the 2,7-diaminoanthraquinone with a pyridine under an ice bath;
(b3) causing the 2,7-diaminoanthraquinone to react with a nitrogen gas; and
(b4) stirring the 2,7-diaminoanthraquinone at a room temperature for 24 hours in the dark.

20. The preparation method according to claim 17, further comprising a step of:
(c) after the step (a), aminating the 2,7-disubstituted anthraquinone derivative to be bounded with a second side chain having an amino group.

21. The preparation method according to claim 20, wherein the step (c) is reacted at a closed device, at a reaction temperature of 130 to 150° C., under an oil bath and for a reaction time of 30 to 50 minutes.


SEQUENCE LISTING (4 sequences)

SEQ ID NO: 1 - DNA, 23 bp, Artificial
  Primer
gggattgggattgggattgggtt

SEQ ID NO: 2 - DNA, 24 bp, Artificial
  Primer
cccttacccttacccttaccctaa

SEQ ID NO: 3 - DNA, 36 bp, Artificial
  Primer
aatccgtcgagcagagttaaaaggccgagaagcgat

SEQ ID NO: 4 - DNA, 18 bp, Artificial
  Primer
atcgcttctcggcctttt